(19) United States
(12) Patent Application Publication
(10) Pub. No.: US 2005/0240352 A1
(43) Pub. Date: Oct. 27, 2005

(54) Online procurement of biologically related products/services using interactive context searching of biological information

(75) Inventor: Feng Liang, San Diego, CA (US)

Correspondence Address: DLA PIPER RUDNICK GRAY CARY US, LLP, 1625 MASSACHUSETTS AVENUE, NW, SUITE 300, WASHINGTON, DC, 20036-2247, US

(73) Assignee: Invitrogen Corporation, Carlsbad, CA (US)

(21) Appl. No.: 10/830,074

(22) Filed: Apr. 23, 2004

(51) Int. Cl. G06F 19/00; G01N 33/48; G01N 33/50

(52) U.S. Cl. 702/19

(57) ABSTRACT

Systems and methods for procuring biologically related products available on a vendor Website are described which involve user-server interfacing with a Web based browser to retrieve database files representing available target products via processing biological context searches on named annotated text string databases.

### BACKGROUND OF THE INVENTION

1. Field of the Invention

The invention relates generally to linking biological information to E-commerce through effective information browsing, processing and reporting, and more particularly, to systems and methods for efficiently searching and extracting relevant data, and for performing contextual data searches on host databases comprising biological content and an inventory of products and/or services indexed as annotated text strings, such as biological sequence databases and databases cataloging other associated biologically related attributes, for the provision of services and/or biologic materials using digital communication.

2. Background Information

With the increasing popularity of computers (for example, personal computers including smaller devices with computing ability) and advancements in telecommunication network technology, many industries have used these new innovations to improve many commercial operations. In the retail-merchandising arena, for example, hosts of products such as books, music, electronics, athletic gear, etc. are available for online purchases through the Internet. By effectively utilizing virtual stores, merchants streamline purchasing and delivery process for both the consumer and retailer. In similar fashion, telecommunication networks make it possible for many other industries to conduct business in a more efficient manner. To name just a few examples, industries taking advantage of such innovations are financial institutions, travel agencies, and news/media networks. In short, a wide range of industries benefit from the use of computer technology to improve communications, regulatory compliance, manufacturing schedules, security, marketing, sales, and distribution of products and information.

As such, the World Wide Web (WWW) has become a significant new medium for commerce, which is referred to as electronic commerce or E-commerce. Vendors offer goods and services for sale via various WWW sites. However, many of the initial WWW systems were not interactive, and typically addressed only ongoing relationships previously worked out manually, for which extremely expensive custom systems needed to be developed at buyers' or vendors' sites.

Many non-commercial Web devices, such as chat rooms and bulletin boards are interactive, each essentially allows two or more people to have conversations over the Internet, in the same way they might speak over the telephone or several might speak over an old-fashioned party line telephone or more recently, participate in a conference call. While the chat room or bulletin board may store these conversations, no other action beneficial to the people involved takes place as a result of the process.

Extranet Web technology has been developed to enable a corporation to “talk to” its suppliers and buyers over the Internet or otherwise secure communication routes as though the other companies were part of the corporation's internal “intranet.” This information exchange is done by using, for example, client/server technology, Web browsers, and hypertext technology used in the Internet, on an internal basis, as the first step towards creating intranets and then, through them and connections to the outside, extranets.

For corporations that sell and distribute at wholesale or retail, one technique for selling goods over the Internet uses the concept of a catalog Website that enables buyers to browse through Web pages and use a “shopping cart” feature for selecting items to purchase. Most of these catalog Websites are significantly limited in the interaction, if any, they allow between buyers and sellers (e.g., U.S. Pat. No. 5,117,354). Many corporations, such as General Electric and General Motors, use electronic communications for soliciting bids and ordering parts, supplies, raw materials, products and services on a wholesale basis. The present system and methods are amenable to any scale and any stage of providing information and ordering products and/or services.

Many vendors of biologically related products have also taken advantage of E-commerce to sell goods and services to buyers. Scientists, as consumers of such products, may be interested in more information about a particular product's characteristics beyond availability and price, to include biological attributes such as sequence similarity, linkage data, metabolic ans signal pathway participation, compatibility with other systems or molecules, alternative pathways for substrate or product (and availability or provision thereof), etc.

For thousands of years, humans, for example, scientists, have been collecting biological data on different types of organisms, ranging from bacteria to human beings. Presently, much of the data collected is stored in one or more databases shared by scientists around the world. For example, a genetic sequence database referred to as the European Molecular Biology Lab (EMBL) gene bank is maintained in Germany. Another example of a genetic sequence database is Genbank, and is maintained by the United States Government.

Another useful database is known as the GO or Gene Ontology database, maintained by the Gene Ontology Consortium. The goal of the Gene Ontology™ (GO) Consortium is to produce a controlled vocabulary that can be applied to all organisms even as knowledge of gene and protein roles in cells is accumulating and changing. GO provides at present three structured networks of defined terms to describe gene product attributes. GO is one of the controlled vocabularies of the Open Biological Ontologies.

Biologists currently waste a lot of time and effort in searching for all of the available information about a desired small area of research. The search is hampered further by the wide variations in terminology that may be common usage at any given time, and that inhibit effective searching by computers as well as people. For example, if one were searching for new targets for antibiotics, he or she might want to find all the gene products that are involved in bacterial protein synthesis, and that have significantly different sequences or structures from those in another organism such as humans. But if one database describes these molecules as being involved in ‘translation’, whereas another uses the phrase ‘protein synthesis’, it will be difficult for an individual—and even harder for a computer—to recognize functionally equivalent terms.

The Gene Ontology project is a collaborative effort to address the beneficial need for consistent descriptions of gene products across different databases. The project began as a collaboration between three model organism databases: FlyBase (Drosophila), the Saccharomyces Genome Database, and Mouse Genome Database (MGD) in 1998. Since then, the GO Consortium has grown to include many databases, including several of the world's major repositories for plant, animal and microbial genomes. Such databases include The Arabidopsis Information Resource (TAIR); the WormBase; the EBI GOA project (i.e., annotation of UniProt Knowledgebase (Swiss-Prot/TrEMBL/PIR-PSD) and InterPro databases); Rat Genome Database (RGD); DictyBase (i.e., informatics resource for the slime mold Dictyostelium discoideum); GeneDB S. pombe; (part of the Pathogen Sequencing Unit at the Wellcome Trust Sanger Institute); GeneDB for protozoa; (part of the Pathogen Sequencing Unit at the Wellcome Trust Sanger Institute); Genome Knowledge Base (GK) (i.e., a collaboration between Cold Spring Harbor Laboratory and EBI); TIGR; Gramene; (i.e., a comparative mapping resource for monocots); Compugen and the Zebrafish Information Network (ZFIN).

The GO collaborators are currently developing three structured, controlled vocabularies (ontologies) that describe gene products in terms of their associated biological processes, cellular components and molecular functions in a species-independent manner. There are three separate aspects to this effort: first, to write and maintain the ontologies themselves; second, to make associations between the ontologies and the genes and gene products in the collaborating databases, and third, to develop tools that facilitate the creation, maintenance and use of ontologies.

The use of GO terms by several collaborating databases facilitates uniform queries across them. The controlled vocabularies are structured so that one can query them at different levels: for example, one can use GO to find all the gene products in the mouse genome that are involved in signal transduction, and one can zoom in on all the receptor tyrosine kinases. This structure also allows annotators to assign properties to gene products at different levels, depending on how much is known about a gene product.

The information content available in one or more of such databases, combined with other information that can be provided by the vendor, can be invaluable to a seeker of information, for example, a buyer interested in the selection of the appropriate biologically related product.

As buyers of such products tend to be more sophisticated users of computer related technologies, and given the wealth of information available in various collections and combinations of biological data, advantages and efficiencies can be obtained from a merging of such biological data with searchable vendor based browsers for biologically related product and service acquisition.

The present invention satisfies this need and provides additional advantages.

### SUMMARY OF THE INVENTION

The present invention relates to methods of accessing biological content and their biologically related products and/or services using one or more electronic inventory files, preferably stored on a compact electronic storage medium. For example, an inventory file is stored on one or more electronic storage media, which may include a number of target items that are separated into various groupings according to their informational format and/or content. In one embodiment, the method includes interfacing by a user or client by way of user terminals and bi-directional communication connections with a server which includes or accesses the electronic storage medium. Further, extracts, which include biological attribute annotations, are generated in the server for each target item stored on the medium by inputting an appropriate request, subsequently the extracts may be retrieved.

Such extracts may contain, but are not limited to, separate categories having one or more data registries or loci which correspond to, for example, headings for organisms, nucleotide accession numbers, related accession numbers, gene names, gene definitions, gene symbols, text summary of gene products, expression profiles, mRNA records, references, length of inserts in base pairs, nucleic acid sequences, collection names, collection types, vector names, vector antibiotics, host names, Stealth RNA, siRNA, protein accession numbers, protein records, amino acid sequences, molecular weights, isoelectric points, protease digestion patterns, domain searches, predicted secondary and tertiary structures, binding sites, classes of enzymes, classes of substrates, associated proteins (for example, other members of protein complexes), inhibitors, blockers, agonists, antagonists, labels, tags, markers or other indicators, protein model searches, Online Mendelian Inheritance in Man (OMIM) data, product data, metabolic pathway data, single nucleotide polymorphism (SNP) data, SNP map data, locus link ID, Unigene ID and genomic alignment data.

In a related aspect, the target server automatically upon request generates an extract based on the content of an associated target item.

In a related aspect, the loci are associated with annotations or objects which provide hyperlinks to one or more internal and/or external database servers.

The resulting outputs from such methods are displayed as browser pages containing for example, hierarchical menus that are based on the retrieved extracts which provide the user with one or more subsets or compilations of the stored target items. The menus represent assortments of target items within the subsets, where the content and/or format of the displayed target items is based on an empirical measure of similarity of the associated biological attributes for all of the assorted target items. Moreover, the hierarchical menu output display pages identify favored or all target items assorted into each of the files which have one or more associated biological attributes in common to enable a user, for example, to differentiate products and/or services of interest stored on electronic media and to obtain or purchase one or more listed products or services (i.e., custom order, catalog listing or service provided) by activating an appropriate graphic user interface (e.g., a check box) that is included on the displayed output pages. In one aspect, any one menu item output on the displayed format page will contain a buy option graphic user interface (GUI) and one or more of the following, including a clone identification number, definition of the expressed product, gene symbol, and accession number.

In a related aspect, the biologically related products include, but are not limited to, cloned nucleic acid inserts comprising one or more items selected from, for example, an open reading frame, structural gene or transcriptional unit, enzymes, buffers, substrates, cofactors, indicator molecules, bioassay, vectors, antibodies, peptides, synthetic nucleic acid, such as DNA and RNA primers and proteins.

In one aspect, each searchable file for a target item includes, but is not limited to, a unique dataset of named annotated text strings having set elements such as a unique name, or identifier, one or more base texts, biologically related annotations that apply to the base text, and/or gene ontology categories. In a related aspect, the ontology category is selected from the group consisting of a biological process, cell component, and/or molecular function.

In one embodiment, the request may include, but is not limited to, inputting a parsable biological attribute in a sub-window accessible module for entering one or more keywords, annotations, sequences, or unique identification numbers. Further, such requests may be processed as, for example, word-for-word searches, Boolean searches, proximity searches, phrase searches, truncation searches or a combination of the above. In other embodiments, methods may include processing string searches using a Blast server (including, but not limited to, in-house or external server) or keyword jump navigation. Further, such searches may include accessing external databases/servers.

In a related aspect, such request may be input by a variety of means, including but not limited to, manual input devices or direct data entry devices (DDEs). For example, manual devices may include, keyboards, concept keyboards, touch sensitive screens, light pens, mouse, tracker balls, joysticks, graphic tablets, scanners, digital cameras, video digitizers and voice recognition devices. DDEs may include, for example, bar code readers, magnetic strip codes, smart cards, magnetic ink character recognition, optical character recognition, optical mark recognition, and turnaround documents. In one embodiment, an output from a gene or a protein chip reader my serve as an input signal.

In another related aspect, the biological attributes may include, but are not limited to, nucleic acid or amino acid sequences, molecular weights, isoelectric points, metabolic and signal pathway participation, restriction maps, organisms, protease fragments, epitopes, hydropathic profiles, separation patterns, such as electrophoresis gels, chromatographic output, mass spec output, fluorescence data, tissue distributions, expression patterns, kinetic constants, binding constants, antagonists, agonists, inverse agonists, linkage maps, substrates, ligands, inhibitors, disease associations, alleles, homologies, interacting molecules, biological functions, phosphorylation patterns, sub-cellular localizations, glycosylation patterns, post-translational modification patterns, motif consensus, crystal structures, pharmacokinetic properties, pharmacologic properties, toxicologic properties, secondary, tertiary and/or quaternary structures.

In one embodiment, when a GUI is activated by the user, the activation triggers the content of the page to be transmitted to a purchase database server. Moreover, the purchase server verifies the transmission to be an order for the product associated with the activated GUI, and subsequently, the verified order is assigned a job number or identifier by the purchase server. Further, the purchase server may enter the verified order and store items selected by the user in a shopping cart database, and thereafter, the purchase server may update the shopping cart database preferably in real time to synchronize the shopping cart database with any incoming transmissions.

In a related aspect, a user can be identified by comparing the customer information in the purchase server with previously-stored customer database information and indicate if a match exists between a customer name field on the transmitted data (e.g., personal names, company names, addresses, institutional names, pass codes, passwords, user IDs, etc.) and the previously-stored customer database information stored on the purchase server (names, addresses, preferences, purchase patterns, last visited site dates, last order dates, etc.).

In another related aspect, customer information can be added to the purchase server customer database when there is not a match between the stored information and that contained in a customer name field.

In another embodiment, transmission to the purchase server can be used to identify the user with a unique session identifier, including embedding the unique session identifier in a universal resource locator (URL). The information can be used to store the user activity in the purchase server, and associate such activity with the session identifier.

In another embodiment, a method of offering a product or service to a user in a remote location is envisaged, including remotely providing access to an electronic data server to a user where the server receives input from a user and processes the input to produce a first output, based on interfacing with one or more public consortium databases, where the latter database has one or more databases which are, for example, proprietary to an offerer of the product or service. The user can select one or multiple products or services or a link or description of a product or service to create an extract, where the extract serves as an output for the user, thus, facilitating delivery of a product or service to the user, whether delivery is remote or local to the offerer/user. In a related aspect, the choice of delivery may be that of the offerer or user.

In a related aspect, the first service may be delivering information to the user, where the product may be a data product. Further, Internet link, electromagnetic wave signal, metallic conductor, or fiber optic cable may provide such remote access.

In another related aspect, a packing function may be facilitated by the method as envisaged (e.g., where special packing requirements are necessary).

In another related aspect, the creation of an extract results in the generation of a message, where such a message is transmitted to a recipient other than the user, including transmission to inventory control, to trigger information related to a manufacturing request or schedule. Further, such a message may relate to compliance with an internal corporate procedure or regulation, a governmental procedure or regulation, or a financial control mechanism. Moreover, such a message is envisaged to be transmissible to a sales representative or may be incorporated into a database tracker for understanding user activity related to an offering/promotion.

The method as envisaged can be used with servers that are either in-house servers, public servers or other private servers. For example, the public server may include a government institution, a private institution, a college or university, a consortium or a private individual. Other databases may include data related to inventory, shippers, seasonal or regional requirements, credit history, hazardous products and interactions, notifications associated with making dangerous or hazardous products, warning flags, etc.

Exemplary methods and systems according to this invention are described in greater detail below.

### BRIEF DESCRIPTION OF THE DRAWINGS

FIG. 1. Illustration of networked computer system.

FIG. 2. Illustration of data set entry.

FIG. 3. Window for Shopping Cart/Purchase Order.

FIG. 4. Window for search browser.

FIG. 5. Flow chart for processing search.

FIG. 6. Block diagram of Index File and File Map.

FIG. 7. Illustration of network search flow for Keyword, Sequence and ID FIG. 8. Flow chart for Purchase processing.

FIG. 9. Flow chart for processing keyword search.

FIG. 10. Browser window for Keyword and/or ID search.

FIG. 11. Results window for Keyword search.

FIG. 12. Results window for ID search.

FIG. 13. Browser window for Sequence search.

FIG. 14. Results window for Sequence search.

FIG. 15. Browser window for Ontology search.

FIG. 16. Illustration of network search flow for Gene Ontology searching.

### DETAILED DESCRIPTION OF THE INVENTION

Before the present invention is described, it is understood that this invention is not limited to the particular methodology, protocols, and systems described as these may vary or be substituted arbitrarily as desired. It is also to be understood that the terminology used herein is for the purpose of describing particular embodiments only, and is not intended to limit the scope of the present invention which will be described by the appended claims.

It must be noted that as used herein and in the appended claims, the singular forms “a”, “an”, and “the” include plural reference unless the context clearly dictates otherwise. Thus, for example, reference to “a subset” includes a plurality of such subsets, reference to “a nucleic acid” includes one or more nucleic acids and equivalents thereof known to those skilled in the art, and so forth.

Unless defined otherwise, all technical and scientific terms used herein have the same meanings as commonly understood by one of ordinary skill in the art to which this invention belongs. Although any methods and systems similar or equivalent to those described herein can be used in the practice or testing of the present invention, the methods, devices, and materials are now described. All publications mentioned herein are incorporated herein by reference for the purpose of describing and disclosing the processes, systems and methodologies which are reported in the publications which might be used in connection with the invention. Nothing herein is to be construed as an admission that the invention is not entitled to antedate such disclosure by virtue of prior invention.

As used herein, “procuring,” including grammatical variations thereof, means to obtain, gain, access, receive, acquire, or buy.

As used herein, “appropriate,” including grammatical variations thereof, means capable of being acted on or carrying out an act. For example, an appropriate request or command when inputted into a dialog box would trigger a search of a database to find or identify an object conforming to the request or command (e.g., keyword search to retrieve objects containing the inputted keyword).

As used herein, “biologically related,” including grammatical variations thereof, means associated with life and living processes. For example, anaerobic respiration is a biologically related metabolic action. Protein expression (in vitro) is another example.

As used herein, “electronic storage medium,” including grammatical variations thereof, means space in electronic memory where information is held for later use. For example, this may include, but is not limited to, magnetic tape, CD-ROMS, DVD, optical disks, flash drives, RAM or floppy disk.

As used herein, “electronic inventory,” including grammatical variations thereof, means a digital catalog which corresponds to some or all of the products and or services offered by the vendor.

As used herein, “target item,” including grammatical variations thereof, means data or files to be affected by an action. For example, a target item can be a file name, a word, an image, a text string, a number or a value stored on electronic media that is retrievable upon request by a user.

As used herein, “sundry groupings,” including grammatical variations thereof, means a collection of various data segregated into named files for orderly access of such data from an electronic storage medium.

As used herein, “interfacing,” including grammatical variations thereof, means the method of interaction between a person and a computer, or between a computer and a peripheral device, or between two computers. In a related aspect, user interface would include the environment that permits one to interact with a computer (e.g., World Wide Web, WiFi, browsers, web pages).

As used herein, “user,” including grammatical variations thereof, means an entity that requests services from a server. The entity can be a human or a device (e.g., see input devices, above).

As used herein, “user terminals,” including grammatical variations thereof, means a node or hardware that accesses a server.

As used herein, “bi-directional communication,” including grammatical variations thereof, means a process by which information is exchanged between two systems in both directions, where each system receives and sends information.

As used herein, “searchable,” including grammatical variations thereof, means the ability of data or files to be looked into in an effort to mark, find or discover such data or files.

As used herein, “extracts,” including grammatical variations thereof, means a product prepared by retrieving files or data from a database or server.

As used herein, “associated biological attributes,” including grammatical variations thereof, means a specific feature related to living things and/or processes of living things (including such a feature carried out in vitro).

As used herein, “request,” including grammatical variations thereof, means one or a series of user inputs or commands for retrieving information from a server or database.

As used herein, “inputting,” including grammatical variations thereof, means the act of entering a request or data. For example, typing at a keyboard pointing, speaking to, etc.

As used herein, “hierarchal menu output,” including grammatical variations thereof, means a list transmitted to the user (e.g., but not limited to, a display on a computer screen) of available alternatives for selection by the operator or user organized into orders or ranks each subordinate to the one above it.

As used herein, “display,” including grammatical variations thereof, means what a user sees on a CRT unit or monitor. More broadly, substitutes may be used as displays, such as auditory signals for the visually impaired or any other means of information communication.

As used herein, “subset,” including grammatical variations thereof, means a set each of whose elements is an element of an inclusive set.

As used herein, “empirical measure of similarity” including grammatical variations thereof, means a method of comparing target items or objects between extracts containing such items or objects, where the extracts are considered to be similar if the distance between the items or objects comprising the extracts is small according to arbitrary values of attributes or annotations associated with items or objects in the target file. For example, values can be given for molecular weights, isoelectric points, metabolic pathway participation, restriction maps, organisms, protease fragments, epitopes, hydropathic profiles, separation patterns, such as electrophoresis gels, chromatographic output, mass spec output, fluorescence data, tissue distributions, expression patterns, kinetic constants, binding constants, antagonists, agonists, inverse agonists, linkage maps, substrates, ligands, inhibitors, disease associations, alleles, homologies, interacting molecules, biological functions, phosphorylation patterns, sub-cellular localizations, glycosylation patterns, post-translational modification patterns, motif consensus, crystal structures, pharmacokinetic properties, pharmacologic properties, and toxicologic properties secondary, tertiary and/or quaternary structures. Thus, for example, each attribute can be given a numerical value. Further, each biologically related product, for example, would have a different set of values for some or all of these attributes/annotations. Extracts with values for one or more attributes/annotations that are numerically similar are judged to be similar. Using such similarity, as distances between values become greater, the extracts are judged as less similar. Based on software design choices, ranks for the spectrum of similarity are determined and the resulting output of the extracts of interest are reflected in hierarchical fashion according to high and low values of similarity. Systems for determining such similarity are disclosed in, for example, U.S. Pat. No. 5,835,087, herein incorporated by reference.

As used herein, “graphic user interface (GUI),” including grammatical variations thereof, means a user interface to a computer that uses icons to represent items, such as documents and programs, that the user can access and manipulate with a pointing device or other signal transducer.

As used herein, “annotated text strings,” including grammatical variations thereof, means text or embedded comments or instructions within text which may or may not print but which may be viewed and referred to by an operator or user that include a consecutive series of characters to be specified by command.

As used herein, “base text,” including grammatical variations thereof, means the number of different values that can be represented by each digit position (e.g., binary or base 2) that correspond to the body copy on a page.

As used herein, “loci,” including grammatical variations thereof, means a site or one or more digital addresses where related information may be found.

As used herein, “objects,” including grammatical variations thereof, means a searchable element that is a part of a locus. For example, an annotation under an “organism” locus would be considered an object.

As used herein, “hyperlinks,” including grammatical variations thereof, means a pointer within a hypertext document that points (links) to another document, which may or may not be a hypertext document.

As used herein, “server,” including grammatical variations thereof, means a functional unit that provides shared services to workstations/clients/users over a network; for example, a file server, a print server, a mail server. The server may be internal or external, single or multitask.

As used herein, “Web page browser,” including grammatical variations thereof, means a program used to read a file or to navigate through a hypermedia document.

As used herein, “parsable,” including grammatical variations thereof, means to be amenable to analysis where the operands entered with a command create a parameter list in the command processor from the information.

As used herein, “sub-window,” including grammatical variations thereof, means a secondary window that is presented to a user to allow the user to perform a task on the primary browser window. For example, a dialog box is a sub-window.

As used herein, “module,” including grammatical variations thereof, means, a self-contained functional unit which is used with a larger system. For example, a software module is a part of a program that performs a particular task.

As used herein, “word-for-word searching” including grammatical variations thereof, means a keyword or keywords serve as the primary unit that represents the information for which the search is being conducted, where the search systems will search for strings of words, as well as individual words. Such a system will not automatically keep words together as a phrase. Further, a word-for-word searching method would envisage the use of wild cards (i.e., include variant endings to any word request).

As used herein, “Boolean searching,” including grammatical variations thereof, means a search structure that uses the logical operators, AND, OR & NOT, to connect search terms in search statements. The operators tell the database what the relationship is between the search terms. Further, a Boolean searching method would envisage the use of wild cards (i.e., include variant endings to any word request).

As used herein, “proximity searching,” including grammatical variations thereof, means a search structure that uses relative location and distance of query words or characters in a search statement. The location and distance operators (e.g., “near,” “adjacent,” “within”) tell the database what the relationship is between the search terms. Further, a proximity searching method would envisage the use of wild cards (i.e., include variant endings to any word request).

As used herein, “phrase searching,” including grammatical variations thereof, means keywords serve as the primary unit that represents the information for which the search is being conducted, where the search systems will search for strings of words. Such a system will automatically keep words together as a phrase. Further, a phrase searching method would envisage the use of wild cards (i.e., include variant endings to any word request).

As used herein, “truncation,” including grammatical variations thereof, means a searching system that uses a symbol at the end of a word to retrieve variant endings of that word.

As used herein, “keyword jump,” including grammatical variations thereof, means a method of navigation that transports a user to content/record stored on a database by entering a keyword or code associated with that content/record.

As used herein, “Blast server,” including grammatical variations thereof, means Basic Local Alignment Search Tool, which is a set of similarity search programs designed to explore all of the available sequence databases regardless of whether the query is protein or nucleic acid.

As used herein, “gene ontology,” including grammatical variations thereof, means a controlled and dynamic vocabulary that can be applied to all organisms as knowledge of gene and protein roles in cells accumulates and changes.

As used herein, “public consortium,” including grammatical variations thereof, means an individual or group recognized by a community to possess authority that can be cited freely by members of the public and understood by members of the community.

As used herein, “tabbed,” including grammatical variations thereof, means a way of creating DHTML dialog boxes, or the like (HTML, XHTML, XML), or sub-windows as a type of interfacing to load such sub-windows.

As used herein, “triggers,” including grammatical variations thereof, means to initiate, actuate, or set off a program.

As used herein, “tree navigation,” including grammatical variations thereof, means using an organization of directories (or folders) and files which resemble the branches of an upside-down tree that allow users to find their way through a Web site.

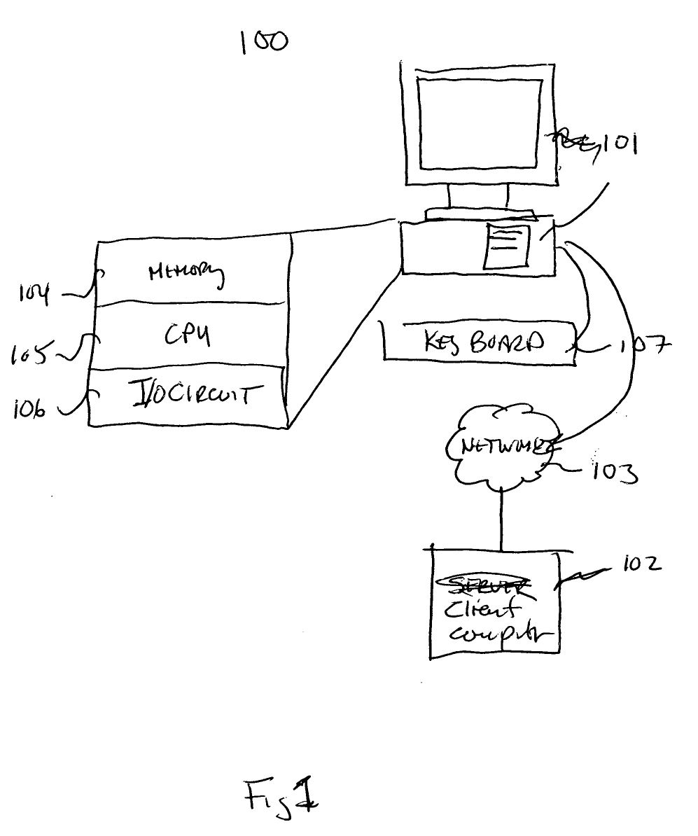

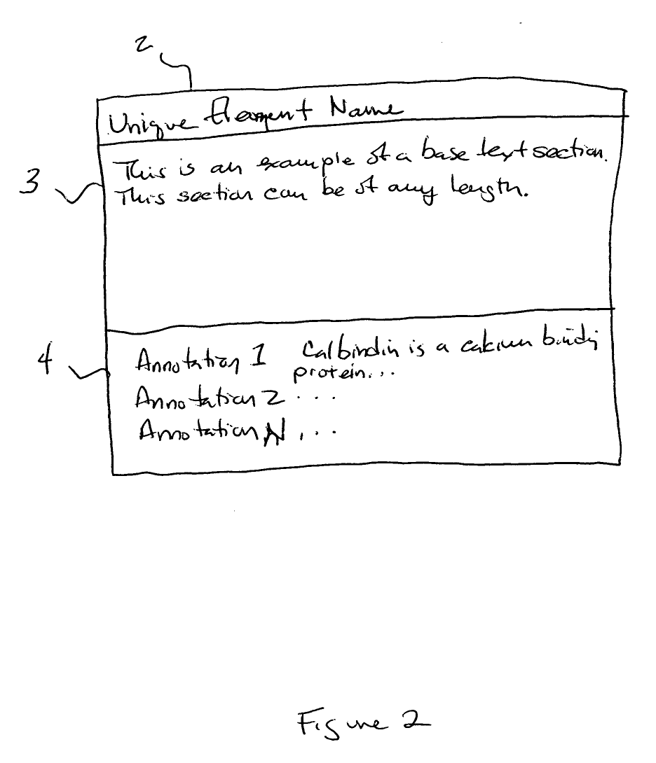

It will be appreciated by one of ordinary skill in the art that computer 101 can be part of a larger system (FIG. 1). For example, computer 101 can be a server computer that is in data communication with other computers. As illustrated in FIG. 1, computer 101 is in data communication with a client computer 102 via a network 103, such as a local area network (LAN) or the Internet.

In particular, computer 101 can include session tracking circuitry for performing session tracking from inbound source to net sale in accordance with the teachings of the present invention. In one embodiment, as will be appreciated by one of ordinary skill in the art, the present invention can be implemented in software executed by computer 101, which is a server computer in data communication with client computer 102 via network 103 (e.g., the software can be stored in memory 104 and executed on CPU 105), as further discussed below.

The present invention may be implemented using hardware, software or a combination thereof and may be implemented in a computer system or other processing system. In fact, in one embodiment, the invention is directed toward a computer system capable of carrying out the functionality described herein. An example computer system 100 is shown in FIG. 1. The computer system 100 includes one or more processors. A processor can be connected to a communication bus. Various software embodiments are described in terms of this example computer system. After reading this description, it will become apparent to a person skilled in the relevant art how to implement the invention using other computer systems and/or computer architectures.

Computer system 100 also includes a main memory, e.g., 104, preferably random access memory (RAM), and can also include a secondary memory. The secondary memory can include, for example, a hard disk drive and/or a removable storage drive, representing a floppy disk drive, a magnetic tape drive, an optical disk drive, memory card etc. The removable storage drive reads from and/or writes to a removable storage unit in a well-known manner. A removable storage unit includes, but is not limited to, a floppy disk, magnetic tape, optical disk, etc. which is read by and written to by, for example, a removable storage drive. As will be appreciated, the removable storage unit includes a computer usable storage medium having stored therein computer software and/or data.

In alternative embodiments, secondary memory may include other similar means for allowing computer programs or other instructions to be loaded into computer system 100. Such means can include, for example, a removable storage unit and an interface device. Examples of such can include a program cartridge and cartridge interface (such as that found in video game devices), a removable memory chip (such as an EPROM, or PROM) and associated socket, and other removable storage units and interfaces which allow software and data to be transferred from the removable storage unit to computer system 100.

Computer system 100 can also include a communications interface (106). Communications interface allows software and data to be transferred between computer system and external devices. Examples of communications interface can include a modem, a network interface (such as an Ethernet card), a communications port, a PCMCIA slot and card, etc. Software and data transferred via communications interface are in the form of signals which can be electronic, electromagnetic, optical or other signals capable of being received by communications interface. These signals are provided to communications interface via a channel. This channel carries signals and can be implemented using wire or cable, fiber optics, a phone line, a cellular phone link, an RF link and other communications channels.

In this document, the term “electronic storage medium” is used to generally refer to media such as removable storage device, a hard disk installed in hard disk drive, and signals. These computer program products are means for providing software to computer system 100.

Computer programs (also called computer control logic) are stored in main memory and/or secondary memory. Computer programs can also be received via communications interface. Such computer programs, when executed, enable the computer system to perform the features of the present invention as discussed herein. In particular, the computer programs, when executed, enable the processor to perform the features of the present invention. Accordingly, such computer programs represent controllers of computer system 100.

In an embodiment where the invention is implemented using software, the software may be stored in a computer program product and loaded into computer system 100 using removable storage drive, hard drive or communications interface. The control logic (software), when executed by the processor, causes the processor to perform the functions of the invention as described herein.

In another embodiment, the invention is implemented primarily in hardware using, for example, hardware components such as application specific integrated circuits (ASICs). Implementation of the hardware state machine so as to perform the functions described herein will be apparent to persons skilled in the relevant art(s).

In yet another embodiment, the invention is implemented using a combination of both hardware and software. In addition, the data computer system preferably includes a display, which can be any device for displaying (101) information in a graphical form, a keyboard (107), which can be any device for inputting characters, and a mouse with a button, which can be any device for indicating screen position.

As envisaged by the present invention, the computer system possesses a database. A database may include, but is not limited to, fields of searchable data, author and title information; textual fields that include biologically related annotations or perhaps the full text; contact fields that include all the bibliographic information and text strings for sequence data. In a related aspect, the choice of properties possessed by particular fields may include fields which are searchable and displayable or displayable only.

In a related aspect, the database is parsable. Parsing is the manner in which information is divided for searching. In a further related aspect, parsing may be viewed in at least one of two ways. One way is word-for-word (word parsing) where the computer breaks at every space. For example, with a title such as “The Electronic Mail Box,” the computer would break after “The,” “Electronic,” “Mail,” and “Box.” Thus, each word would be searchable. Further, with word parsing systems, the computer can be programmed to ignore words such as “the,” “of,” and, “but,” etc. Moreover, a hyphenated word may be read as a single word by the computer, so the text must be impeccably consistent if the system is to operate effectively.

A second method is phrase parsing. In this system, the breaks occur only where indicated “break.” The break indicator, or subfield delimiter, determines where each phrase is to be broken. Phrase parsing solves the problem of double-word descriptors. Within these breaks the information must be consistent in order to facilitate searching. Also, as envisaged by the present invention, a system can be programmed for both word and phrase parsing to make searching more extensive and complete.

Alternatively, a Boolean expression may be supplied by the user to retrieve files from the database (see, e.g., U.S. Pat. No. 4,384,325). For example, such an expression would involve a process of arithmetically comparing fields of records within a database to corresponding fields of records containing reference words in order to derive arithmetic, logical comparisons. The comparison results would be compared to inputs of a user supplied Boolean expression (e.g., those that contain AND, OR, AND NOT, etc.) to determine if the comparisons satisfy the user supplied Boolean expression. In one embodiment, there would be a corresponding indication where a Boolean expression hit is determined based on identification of an appropriate record and a separate indication as a Boolean expression miss whenever the Boolean expression is not satisfied upon determining the comparison.

The present invention may be embodied in a software program residing on a data processing system operating under Unix and/or Windows operating systems. In one embodiment, the software program is written in the per, C, C++, C# and Java programming languages and uses the relational database management system, as the data storage.

According to the present invention, the data processing system receives a query, such as a natural language query, from a user and displays the terms of the query on a display screen. Each term is preferably displayed surrounded by a box. A displayed term and its surrounding box is called a “tile,” although the term “tile” should not be limited only to the use of a box surrounding a term. Instead, a “tile” refers more generally to a graphical representation corresponding to a displayed query term.

The data processing system, as envisaged, also preferably includes a dictionary and a thesaurus stored in another auxiliary memory, which is preferably an external hard disk drive, but could also be an external CD ROM or similar device. The dictionary contains a list of words that can be used, for example, as terms in the Boolean query and identifies the part of speech for each of the words. The words may be stored in the dictionary in “citation form,” which is a morphologically uninflected form that is related to a number of variations of the term. For example, the term “copy” may be preferably stored in the dictionary and identified as either a verb or a noun. The memory includes morphological rules to change words such as “copied,” “copies,” and “copying” to their citation form of “copy” before they are looked up in the dictionary. Similarly, certain query terms using lower case letters are stored in the dictionary with a citation form having all capital letters. Thus, “sql” would be stored as “SQL.” Such a system maintains a list of morphological rules for shortening words to their citation forms in memory and a list of parse rules for syntactic analysis in memory.

Target items and queries may be associated with tags as flags for generating and sending notices, such as a single flag to trigger notification of non-user managers/systems (e.g., sales, manufacturing, news release, IT maintenance and security, accounting, financial management or support etc.). In a related aspect, multi-flag notices are envisaged, where a set of flags is associated with target items or queries, which then trigger such notification as above. In a further related aspect, override flags such as not to notify a security function when for example, the query is from a specific source or list of sources. In another related aspect, the multi-flag tagging involves the use of a decision tree to determine which if any of the non-user managers/systems are to be notified.

A thesaurus stores lists of words related to citation terms. The related words preferably include more specialized/more general words, lists of synonyms, alternative terms and lists of related terms. The exact organization of both the dictionary and thesaurus is not important to the present invention. Any organization that will accommodate the invention may be used.

In a related aspect, most files, such as those produced by the large time-sharing vendors, have what is known as a “basic index,” or “default file.” This file index consists of the basic controlled term vocabulary as well as terms preceded by their categorical mnemonics, such as OR for “organism,” NA for “nucleotide accession,” GN for “gene name,” or RF for “references.” In one embodiment, searching can be processed using the mnemonic tags or codes or through general, or natural language terms. In one embodiment, for each index an inverted file is created. The advantage of an inverted file is its speed.

In one embodiment, the database comprises sets of named annotated text strings. Each element of the set is defined (e.g., unique identification, base text, etc.). Annotations can be applied to any element of the set (e.g., base text).

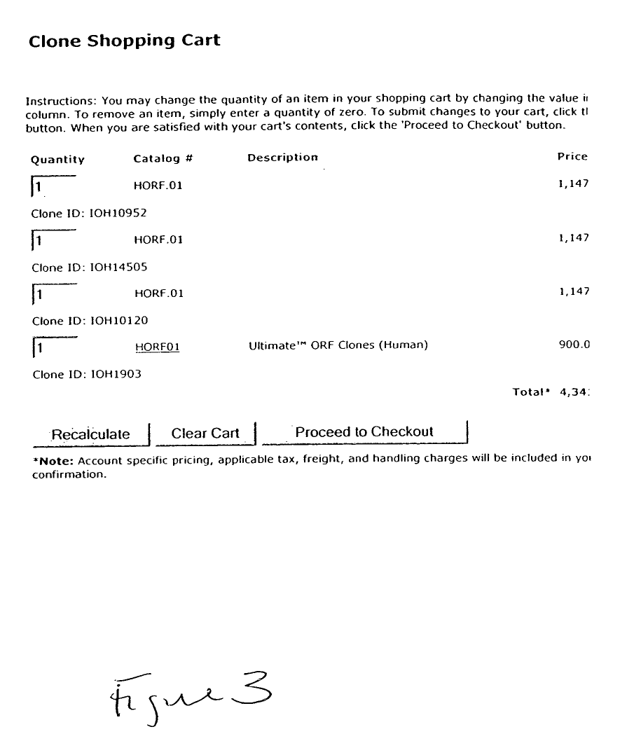

An example of data set entry is illustrated in FIG. 2. The entry 1 comprises a unique element (identification) name 2, a base text section 3, and an annotation section 4.

In another embodiment, further additional indexing may be attached. For example, providing full-text searching in addition to a basic index. Such a full-text search increases the coverage of the search. In a related aspect, the search can be absolutely scoped (limited to only certain parts of a site) or scoped to a topic, category or idea.

“Dialog box” refers to sub-widows that open to provide a user with a set of options from which to choose. The dialog box may contain control options that are split into two or more tabs. Tabs may include, but are not limited to Search By Sequence, Search By Keyword/ID, Browse By Ontology and ORF FAQs (Frequently Asked Questions). Further, the dialog box may contain one or more buttons that present the user with two or more mutually exclusive options. For example, to limit search to human or mouse species for a sequence search, a user may check the appropriate button in the dialog box prior to search.

Right-clicking and shortcut menus are available, to get quick hints about what an item is or what it can do to view its shortcut menu. The short cut menu can offer a list of options e.g., properties, printing, open a new window, save target as, add to favorites, define how item functions and/or proper method of interfacing by user.

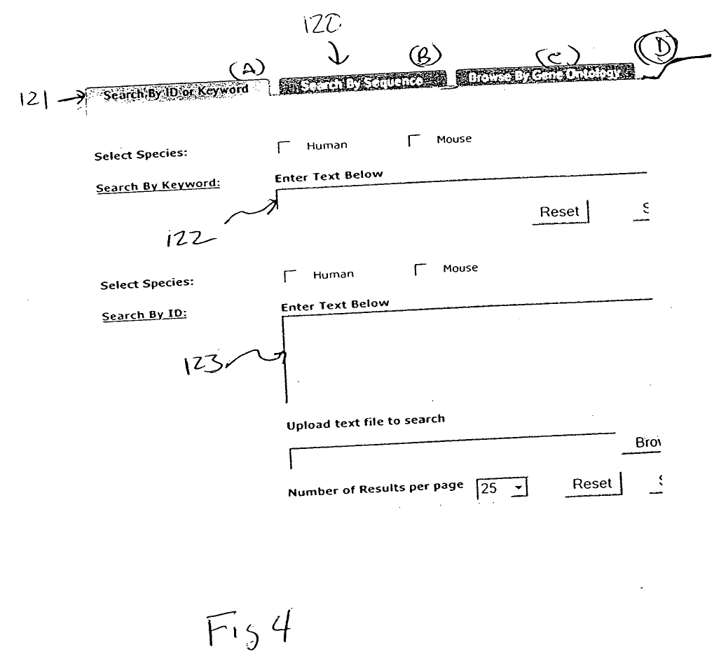

The user interacts with the system through a user interface. A user interface is something which bridges the gap between a user who seeks to control a device and the software and/or hardware that actually controls that device. The user interface for a computer is typically a software program running on the computer's central processing unit which responds to certain user-entered commands. Order entry system (FIG. 3) uses object-based windows as the preferred user interface. In a related aspect, PowerBuilder® by Powersoft Corporation is used as the window development tool.

In one embodiment, the present invention can be implemented using an interactive graphical user interface for specifying and refining database queries. One example of such an interface is provided by the “AVS™” visual application development environment manufactured by Advanced Visual System, Inc., of Waltham Mass. Another example of a visual programming development environment is the IBM® Data Explorer, manufactured by International Business Machines, Inc. of Armonk, N.Y.

It is noted that using a visual-programming environment, such as AVS, is just one example of a means for implementing an embodiment of the present invention. Many other programming environments can be used to implement alternate embodiments of the present invention, including customized code using any computer language available. Accordingly, the use of the AVS programming environment should not be construed to limit the scope and breadth of the present invention.

In one embodiment, using such a system reduces custom programming requirements and speeds up development cycles. In addition, the visual programming tools provided by the AVS system facilitate the formulation of database queries by researchers who are not necessarily knowledgeable about databases and programming languages. In addition, an advantage to using a programming environment such as AVS, is that the system automatically manages the flow of data, module execution, and any temporary data file and storage requirements that may be necessary to implement requested database queries.

AVS is particularly useful because it provides a user interface that is easy to use. To perform a database query, users construct a “network” by interacting with and connecting graphical representations of execution modules. Execution modules are either provided by AVS or are custom modules that are constructed by skilled computer programmers. For example, customized AVS modules can be constructed using a high level programming language, such as C, C++ or FORTRAN, in accordance with the principles as described.

The purpose of constructing a network in AVS is to provide a data processing pipeline in which the output of one module can become the input of another. In one aspect of the present invention, database queries are formulated in this manner. A component of the AVS system referred to as the “Flow Executive” automatically manages the execution timing of the modules. The Flow Executive supervises data flow between modules and keeps track of where data is to be sent. Modules are executed only when all of the required input values have been computed.

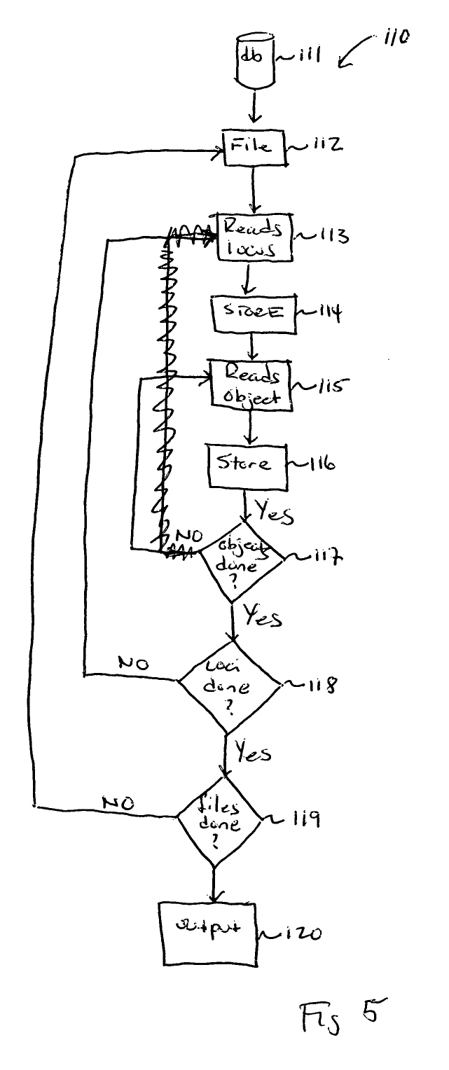

One envisaged user interface is shown in FIG. 4. The user interface employs window 120 preferably in the form of a rectangular shaped box having a toolbar 121 across the top which provides a set of standard menu options represented by a plurality of tabs or buttons A through D.

Window 120 also includes a plurality of other tabs/buttons represented preferably as search options. Tab A typically represent an action or choice which is activated immediately upon user selection thereof. The tabs/buttons on window 120 may contain text, graphics or both. In a related aspect, buttons A through D contain graphics (i.e., icons) so that the user may readily determine the function they represent.

Window 120 preferably includes a plurality of data capture fields 122 and 123 for capturing data. The data capture fields allow the capture of variable length text. The data can be captured either automatically by system-to-system communication or by the user, such as through a keyboard.

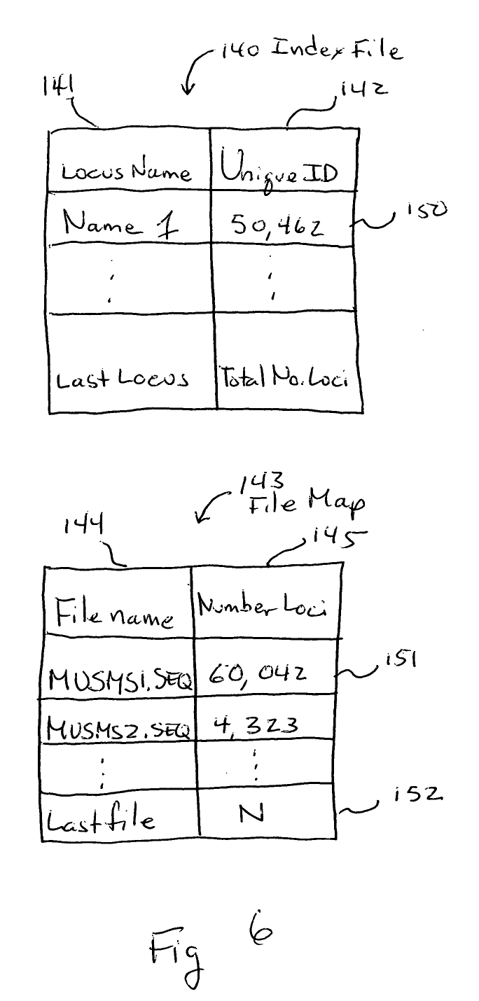

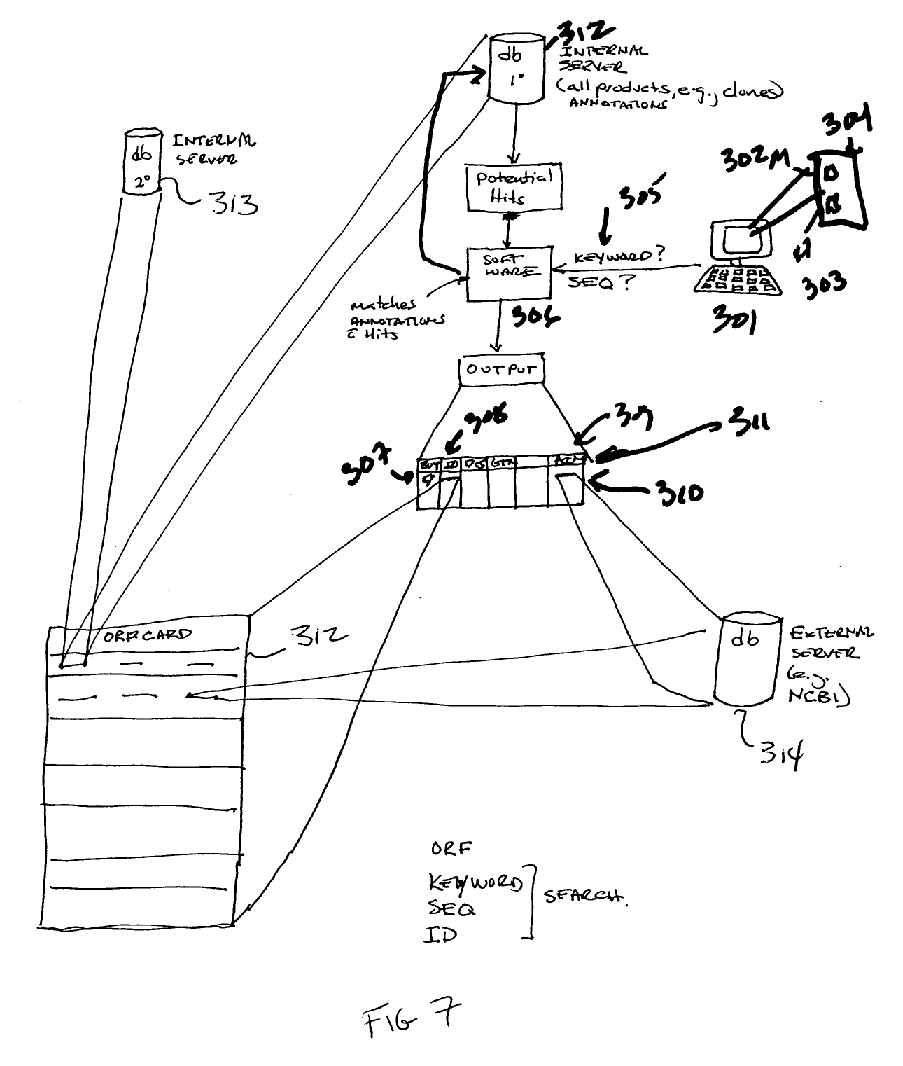

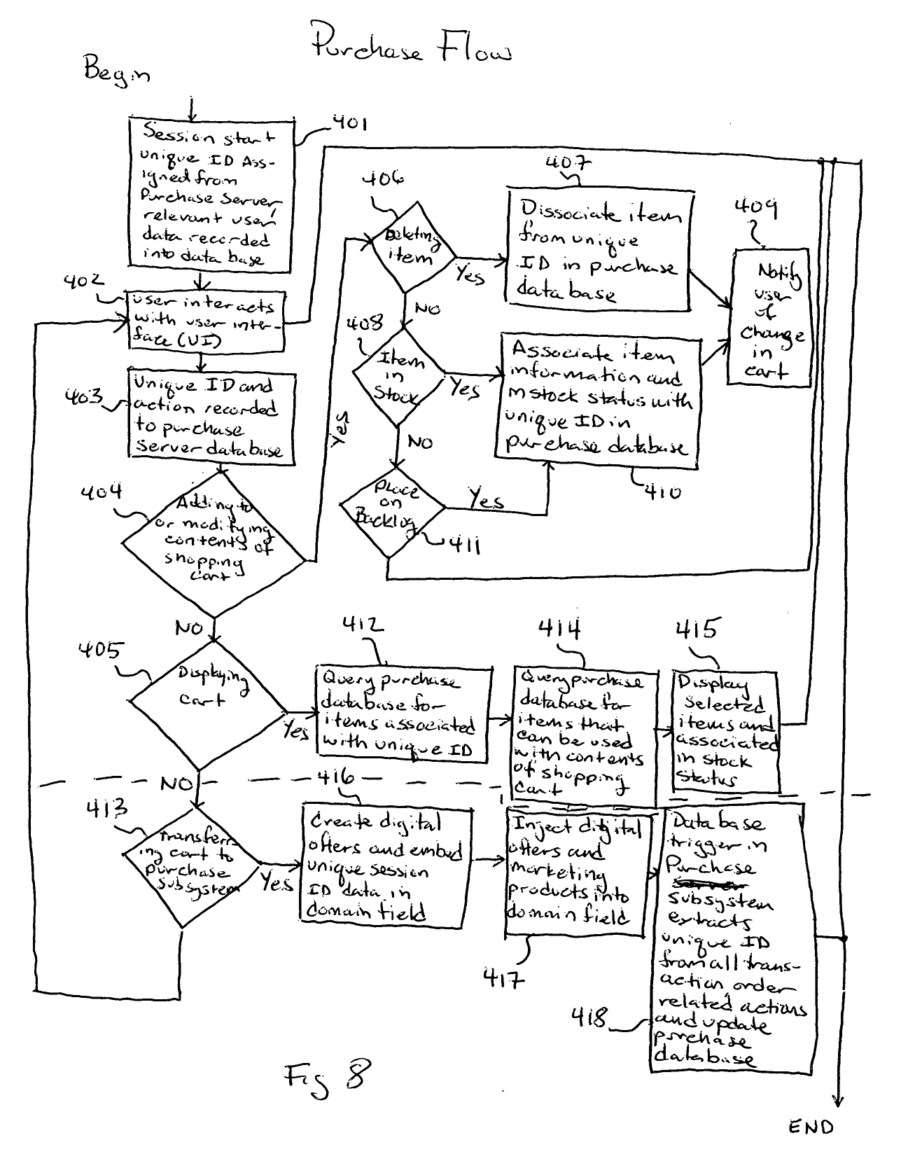

FIG. 5 is a flowchart (110) that depicts the beginning process that can be used to search for a record. The process begins with step 111, where control immediately passes to step 112. In step 112, the process opens the next ORF file. Typically, the first time step 112 is executed, the first file listed in the file map is opened. An example of a file map can be seen in FIG. 6. FIG. 6 illustrates in block diagram form the contents of an index file and a file map in accordance with an embodiment of the present invention.

As shown, the index file 140 comprises, for example, the unique Name 1 of each element in the database (see e.g., FIG. 2), and a unique ID 142 that is assigned to each element. Typically, the unique ID 142 assigned is simply the order number in which the entry appears in the database. Typically, when multiple files are used, their ordering is performed according to the file map described below.

A file map 143 may comprise the file name of each file in the database, and the number of entries (loci) within each file. Thus, given a loci number (i.e., the unique ID 142 assigned to each loci, as described above), one can easily determine which file contains the entry by consulting the file map 143.

Returning to FIG. 5, next, in step 113, the process parses the file and reads the next locus in the file. Of course, the first time step 113 is executed for each file, the first locus in the file is read. Next, as indicated by step 114, the offset and length of the locus read and parsed in step 113 is stored in an associated card file (card files contain a road map pertaining to the searchable objects within the associated locus). Typically, for example, the card file would have same name as the associated sequence file for identification purposes. For example, for a mouse file named “MUSMS.SEQ,” the associated card file is named “MUSMS.CRD.”

Next, as indicated by step 115, the next searchable object is read. For example, the first time this step is executed, the LOCUS section is read and its offset and length are determined. This offset and length is next stored in the associated objects file, as indicated by step 116. Typically, for example, the objects file would have the same file name (but different file type), as the associated sequence file for identification purposes. For example, for a mouse file named “MUSMS.SEQ,” the associated parameter file is named “MUSMS.OBJTS.”

Next, as indicated by step 117, the process determines if there are additional searchable objects in the locus. If so, control loops back and steps 115 and 116 are executed, thereby storing offsets and lengths for all searchable objects in the locus, until all searchable objects have been processed.

As indicated by step 117, once all searchable objects have been processed, control passes to step 118. In step 118, the process determines if there are any additional loci remaining in the file read in step 117. If so, control passes back to step 113, and the next locus is processed in the same manner as described above. Once the last locus in the file has been processed, control passes to step 119, as indicated.

In step 119, the process determines if there are any more files listed in the file map that need to be processed. If so, control passes back to step 112, where the next file is opened. Next, the process repeats itself, as described above, until all files have been processed in the manner described above. Finally, as indicated the process ends with step 120.

The net result of the process depicted in FIG. 5, is the creation of an index file and an objects file (i.e., extract) for each file used in a particular implementation of the present invention.

The index files and object files are each read into memory and a file name is associated for each Unique ID once the system receives a request to perform a search on a particular locus.

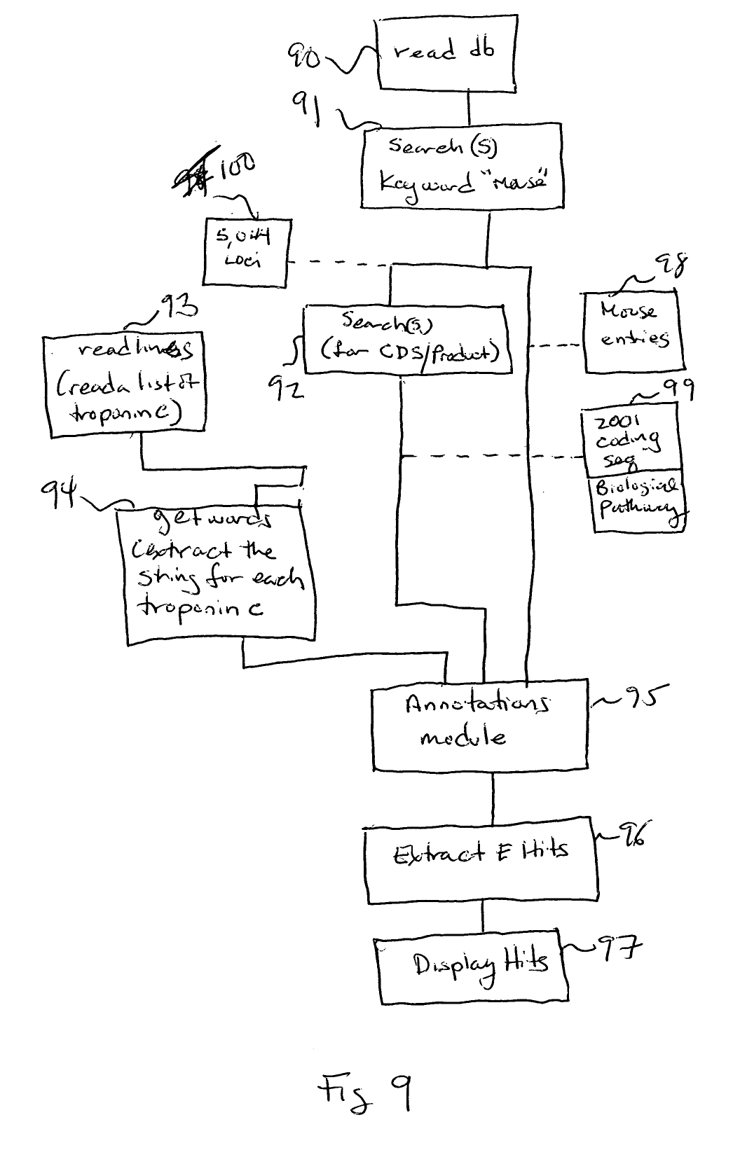

A flow chart for use of the index file and object file is shown in FIG. 7. A user interface 301 allows the user to input parsable/searchable information (e.g., a word, phrase, sequence, ID number). Optionally, the search can be scoped by activating GUI 304 prior to inputting parsable/searchable information 305. In the next step, the scoped search limits access to only a certain portion of all of the products available on the database 302 (e.g., all mouse data, each associated with a unique ID). Software 306 processes the inputted command to limit output to only those files matching the keyword within the scoped products, e.g., page 311.

The output page will contain a list of hits 307 corresponding to the input command, where the user can point to embedded hyperlinks to access annotation data associated with, for example, a unique ID number 308 or accession number 309. If the hyperlink for the unique ID number 310 is activated, the number is used to search the index file and the corresponding data is matched to the objects file. Matching of the index and object file will retrieve the appropriate locus from the ORF file database 312 and an annotated document for the unique ID number will be displayed to the user.

FIG. 8 is a purchase flow diagram of interactive network session tracking from inbound source to net sale in accordance with one embodiment of the present invention. Operation begins at stage 401 in response to a new user initiating access to an interactive network site. At stage 401, a unique session ID (identifier) is assigned from a front-end session database, and relevant user data is recorded in the session database associated with the session ID. For example, the relevant user data includes the user's inbound source (origin), such as a unique source ID of a banner (advertisement) on a search engine WWW site (e.g., which can be determined using standard name-value pairs passed via HTTP protocol).

At stage 402, the user interacts with the user interface of the network site. For example, the user interacts with the WWW online site by adding or deleting items from a virtual shopping cart or by jumping to different, dynamically generated HTML pages of the WWW site. At stage 403, any action performed by the user during stage 402 is recorded in the session database and associated with the session ID.

At stage 404, whether the user added or modified items in the shopping cart during stage 402 is determined. If so, operation proceeds to stage 406. Otherwise, operation proceeds to stage 405. At stage 406, whether an item is to be deleted from the shopping cart is determined. If so, operation proceeds to stage 407. Otherwise, operation proceeds to stage 408. At stage 407, the deleted item is disassociated from the session ID in a purchase server shopping cart database. Operation then proceeds to stage 409, which is discussed below. At stage 408, whether the item to be added is in stock is determined. If so, operation proceeds to stage 410. Otherwise, operation proceeds to stage 411. At stage 410, the added item is associated with the session ID in the shopping cart database. The in-stock status is also associated with the session ID in the shopping cart database. At stage 411, the out-of-stock item is placed on backorder. The entry in the shopping cart database that is associated with the session ID is then appropriately updated at stage 409. At stage 409, the user is notified of the change in the shopping cart. For example, the user is appropriately notified of the added or modified item(s) in the shopping cart.

In one embodiment, if the item is out of stock or the item requires custom service (e.g., but not limited to, antibody generation, clone production, vector design, nucleic acid/primer design, etc.), alternatively, the user can be linked to a product service page for such custom service. Further, the user can be linked directly to a service, technical or customer representative.

At stage 405, whether the user desires to have the contents of the user's shopping cart displayed is determined. For example, the user may want to view the currently added items in the user's shopping cart. If so, operation proceeds to stage 412. Otherwise, operation proceeds to stage 413. At stage 412, the shopping cart database is queried for items associated with the user's session ID. This can include items or services that can be used in connection with contents of the shopping cart (e.g., enzymes, clones, vectors, antibodies that can be used with protein query, custom designs for plasmids, maps, host organisms, etc.). At stage 415, the selected items and associated in-stock status are displayed to the user. For example, the user's selected items for purchase are output to the user's display.

At stage 413, whether the user is ready to purchase the currently selected items is determined. If so, operation proceeds to stage 416 and transitions to a (secure) purchase subsystem (e.g., a purchase subsystem that communicates via the Internet using an encrypted protocol to protect sensitive financial data). Otherwise, operation returns to stage 402. In particular, as shown by the horizontal dashed line of FIG. 8, if the user elects to proceed to purchases of the selected items in the user's shopping cart, then operation transitions across a seam between a first subsystem and a second subsystem of the network site (e.g., a WWW server). In one embodiment, the first subsystem is a catalog subsystem, which uses standard HTTP protocol, and the second subsystem is a secure purchase subsystem, which uses standard SSL (Secure Sockets Layer) protocol (i.e., an encrypted protocol for security purposes).

At stage 417, a digital offer is created to execute a net sale transaction (e.g., a customer order) of the selected items. For example, the shopping cart data stored in the shopping cart database can be passed to Open Market's commercially available TRANSACT software for creation of one or more digital offers (e.g., one digital offer per product). The session ID is embedded in the Domain field (also called the unique ID field) of each digital offer such that inbound source, user activity at the network site, and net sales data are all associated with the same unique session ID for subsequent (e.g., offline) correlation and analysis.

At stage 418, the digital offer is injected into a transaction database, such as the commercially available Open Market TRANSACT database. Thus, the user's shopping cart data is also maintained in the transaction database of the purchase subsystem and is associated with the user's unique session ID.

The user can modify items in the user's shopping cart after entering into the purchase subsystem. For example, the user may decide to delete an item from the user's shopping cart. Accordingly, at stage 418, the shopping cart data associated with the session ID that is stored in the Open Market TRANSACT database is extracted from all TRANSACT order-related actions and the shopping cart database is appropriately updated. Accordingly, the shopping cart database of the catalog subsystem is synchronized with the shopping cart data stored in the transaction database of the purchase subsystem. If the user executes any further interactions with the user interface of the WWW online site, then operation returns to stage 402. Otherwise, (i.e., the user exits the browser session) operation terminates.

In a related aspect, each new record includes the new session ID, a source ID (i.e., an inbound source), a time stamp, a referrer URL (Universal Resource Locator), an IP (Internet Protocol) address, and an entry point (e.g., WWW online site start page). The session ID is associated with the user's browser session using a standard transient (HTTP) cookie (i.e., the cookie stored on the user's computer includes the session ID). Thus, the user's subsequent actions (e.g., HTTP requests) are associated with the user's unique session ID at least until the user exits the user's browser (i.e., the user's session is viewed as the life of the user's browser session).

In one embodiment, such user information can be used to track the accumulation of materials for illicit purposes (e.g., bio-terrorism), where orders to be shipped to separate sites for assembly may be tracked back to the same URL.

In another related aspect, every WWW page (e.g., HTML page) that is viewed is tracked in the session database and associated with the session ID. Further, every shopping-cart-related activity is tracked in the session database and associated with the session ID. In particular, the session database records include the following: the session ID, the time stamp, the page viewed or nature of interaction, and (for shopping-cart-related activities) the online products or services added or modified.

In a further related aspect, when adding a product to the shopping cart, a new record is added in the shopping cart database. For example, the new record includes the session ID, a model identifier, an in-stock indicator (e.g., Y or N for in stock or out-of-stock, respectively, which can then be interpreted to determine if an added item is on back-order), and a quantity. Moreover, when modifying the quantity of an item already in the shopping cart, the record in the shopping cart database containing the item is located using the session ID, model, and in-stock indicator as criteria. The appropriate criteria can then be updated. An adjusted quantity can trigger a change to an out-of-stock indicator if the quantity exceeds available inventory. At stage 406, when deleting a product from the shopping cart, the appropriate record is located as similarly discussed above. The located record can then be deleted.

The following examples are intended to illustrate but not limit the invention.

### EXAMPLE 1

### Advanced Search Modules

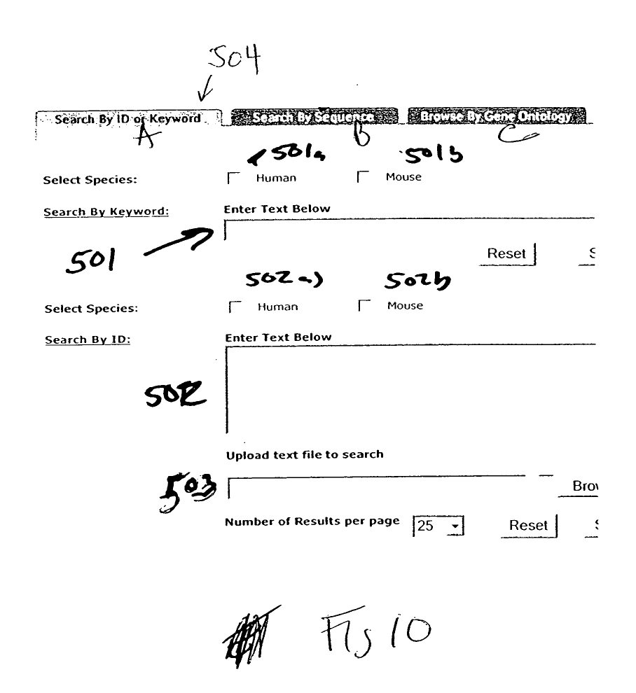

Advanced search modules 120 identify the way in which a user may retrieve objects from the server for that are of procurement interest. A dialog flow for the advanced search modules is shown in FIG. 9.

In FIG. 9 a search is performed in the mouse database to search for troponin C for mice. As shown, the first step is to execute the read database module 90. The output is the mouse portion of the database. Next, as indicated, the search database module 91 is executed. In this case, the user enters search parameters to extract all “mus musculus” (mouse) entries from the database. As indicated by the output block 98, this results in a total of 60,055 entries.

Next, the search database module 92 is again executed. This time the input is the 5,044 mouse loci from module 81. This time the search is performed to find coding sequences (CDS). A read lines module 93 is executed in parallel for reading in a pre-compiled list of named troponin c sequences. Next, as indicated, a get-words module is used to extract the sequence from each of the named troponin C sequences.

Next, the search database module 95 is executed. The search database module 95 has three input parameters. The first input parameter is the Hits list 100 comprising the 5,044 mouse loci. The second parameter is the Hits list 99 comprising the 2001 coding sequences. The coding sequences 99 are used to provide a context to the Annotation module 95. This annotation is used in conjunction with parameters from the vendor that defines the relationship for the annotation. For example, the vendor can specify a search for troponin c sequence 93 that is associated with pathway information 99

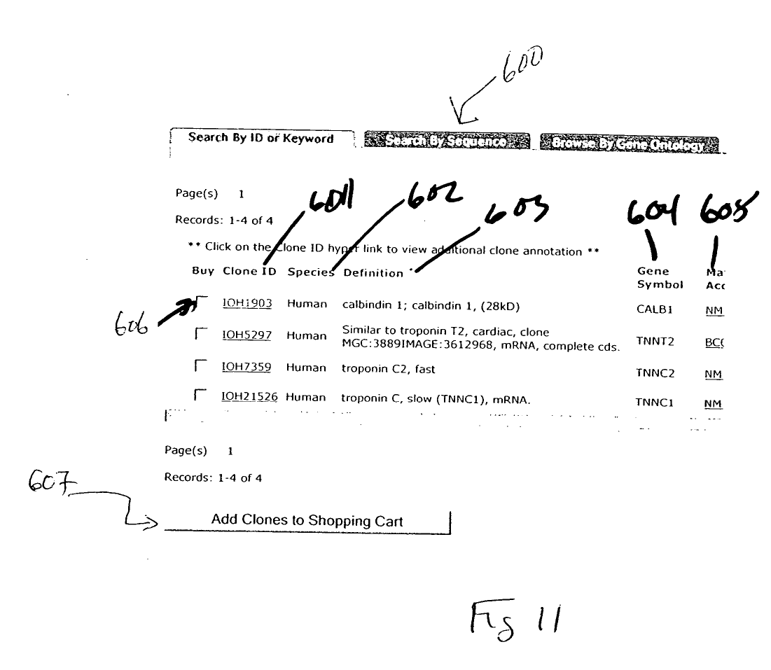

In order to initiate a search, the user must be able to pull up a subset of target items from the system. In this regard, the advanced search modules used are made up of at least 3 functions (FIG. 10), namely Search By Keyword/I.D. (which includes text file searching), Search By Sequence, and Browse By Ontology, all of which may be further parsed by selection of species (501(a) and (b)). These functions may be represented by tabs 504 (A), (B), and (C) of the user interface of FIG. 10. For example, such dialog boxes may include Search By Keyword (to include Select Species buttons 501 (a) and (b)) 501, Search By ID (to include Select species buttons) 502, and Upload text file to search 503.

Search By Keyword

Prior to activation of Search By Keyword 504, buttons are available for selection of species (501 (a) and (b)). Further, the number of results per page can be delimited on the first page of the browser.

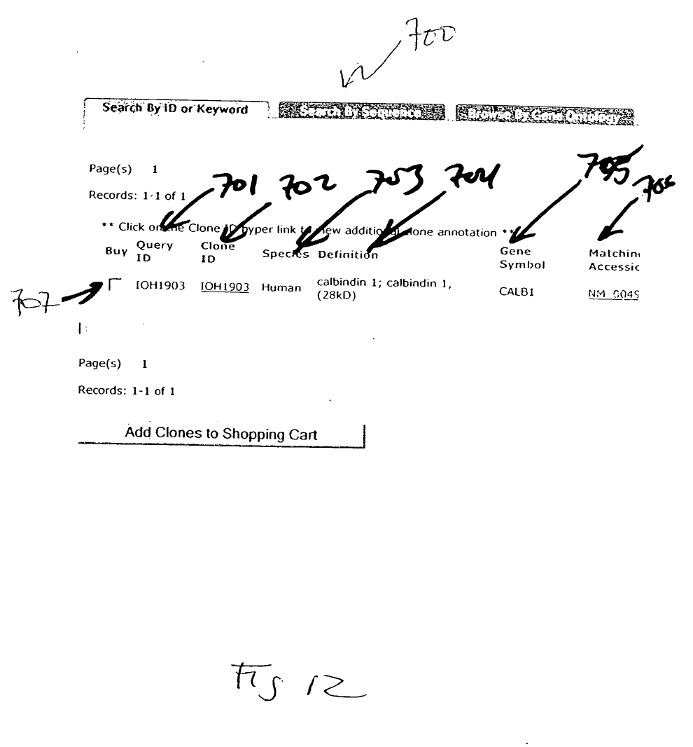

Upon inputting of keywords in the appropriate dialog box, a window 600 as shown in FIG. 11 opens and permits the user to view the products which conform to the biological attributes associated with the keywords. The search results window 600 defines the number of pages and records which conform to the search criteria of the user. As is shown from search results window 600 of FIG. 11, 5 search criteria data fields are preferably identified. These include a Clone ID field 601, species field 602, definition field 603, Gene Symbol filed 604 and Accession Number field 605. Also included is a button for the option to buy the biological material(s) meeting the criteria of the search (606).

It is understood that the search criteria will vary depending upon the keywords and species selected. Upon selecting a keyword and species, window 600 displays at least one page of results representing a number of records associated with the keywords currently used. For example, in the case of troponin C (human), window 600 provides results page displaying the number of pages encompassing the records, the number of records, option to buy, Clone ID, Species, Definition of the clone, Gene Symbol and Accession Number associated with the cloned gene (FIG. 11).

Search by ID

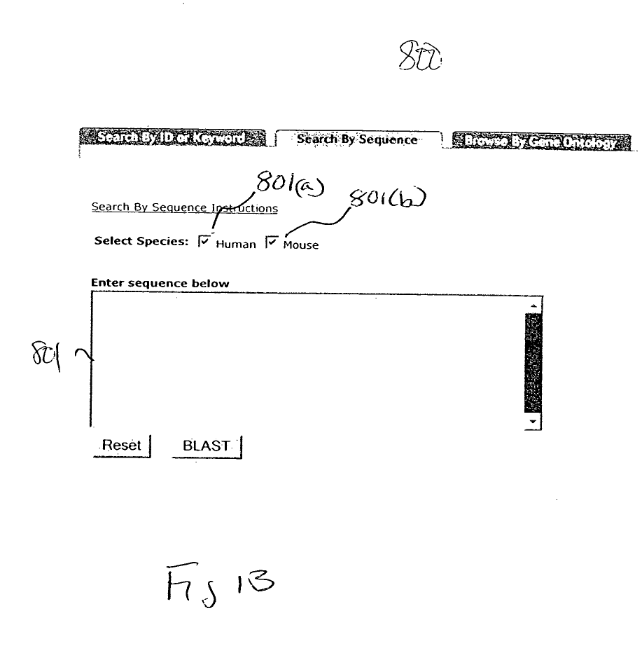

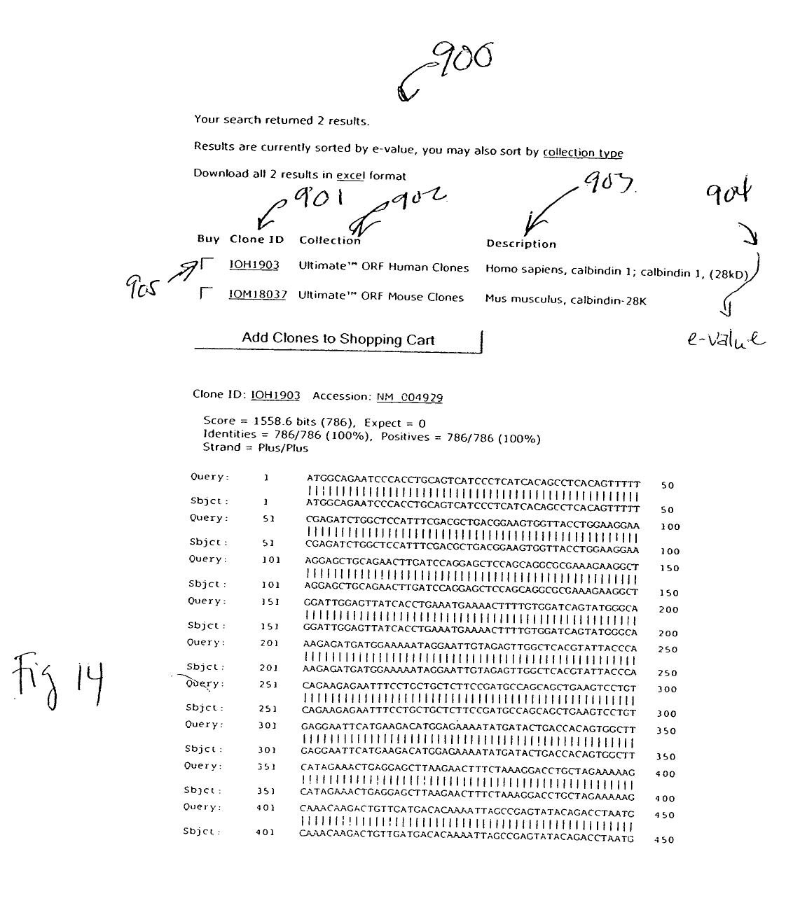

Prior to activation of Search By ID 502, buttons are available for selection of species (502 (a) and (b)). Upon inputting of appropriate ID (e.g., Catalog Number(s), GenBank Accession(s) Gene Symbols(s), LocusLink ID(s), Unigene Cluster ID(s), etc.) in the appropriate dialog box, a window 700 as shown in FIG. 12 opens and permits the user to view the products which conform to the biological attributes associated with the ID numbers. The search results window 700 defines the number of pages and records which conform to the search criteria of the user. As is shown from search results window 700 of FIG. 12, 6 search criteria data fields are preferably identified. These include a Query ID field 701, Clone ID field 702, species field 703, definition field 704, Gene Symbol filed 705 and Accession Number field 706. Also included is a button for the option to buy the biological material(s) meeting the criteria of the search (707).

Again, it is understood that the search criteria will vary depending upon the type of ID used and species selected. Moreover, text files can be uploaded from the users computer to the browser page at the “Upload Text File to Search” field for subsequent search (FIG. 10, 503).

Search by Sequence

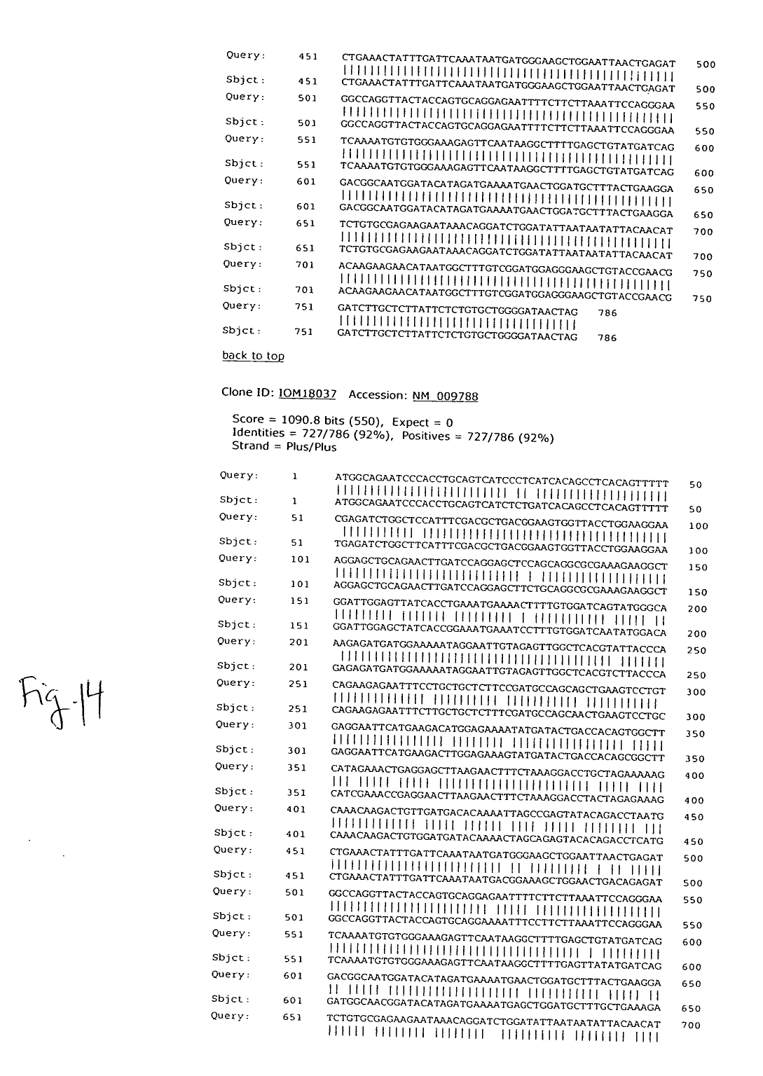

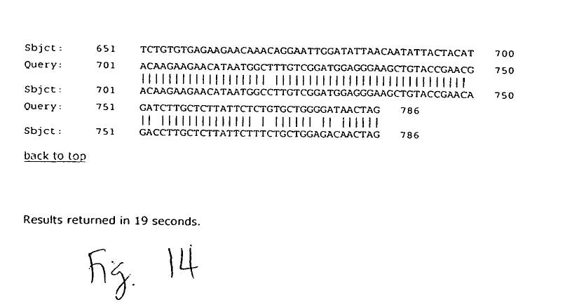

Prior to activation of Search By Sequence, buttons are available for selection of species (FIG. 13, 801(a) and (b)). Upon inputting of appropriate sequence (e.g., the input sequence window accepts nucleotide/amino acid sequences between 50 and 10,000 residues in FASTA, GenBank, and text formats, blastn is used to search the clone databases and results with e-values less than 0.01 are reported, etc.) in the appropriate dialog box (801), a window 900 as shown in FIG. 14 opens and permits the user to view the products which conform to the biological attributes associated with the sequence. The search results window 900 defines the number of results which conform to the search criteria of the user. As is shown from search results window 900, 4 search criteria data fields are preferably identified. These include a Clone ID field 901, collection field 902, description field 903, and e value 904. Further a field is available for linking user to the specific sequence described in 904. Also included is a button for the option to buy the biological material(s) meeting the criteria of the search (905).

Browse by Ontology

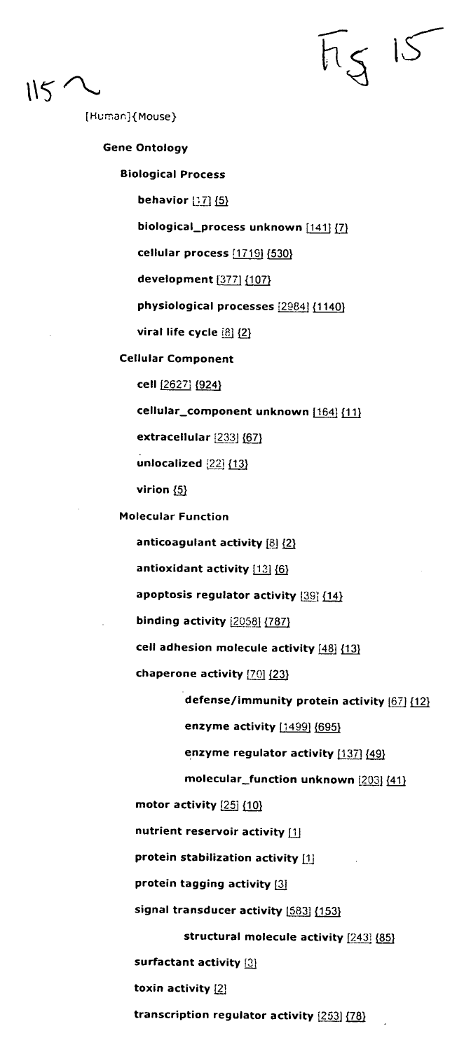

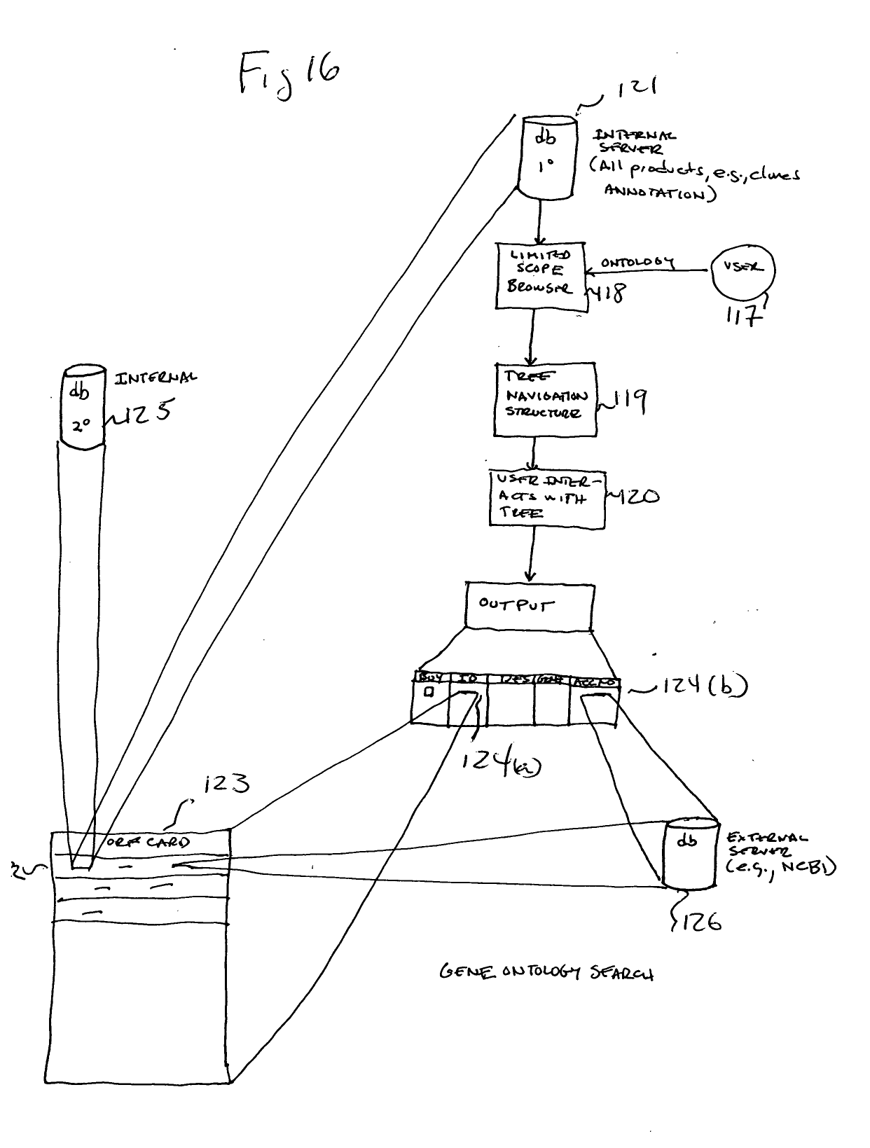

Activation of the Browse by Ontology tab triggers a keyword jump which loads a separate limited scope page (FIG. 15, 115). The illustration in FIG. 16, diagrams the flow (116). Using tree navigation (119), the gene ontology page displays, for example, three categories for viewing/activation by the user (e.g., Biological Process, Cellular Component, or Molecular Function). The user then activates a GUI (e.g., button, 120), that displays a number of headings (behavior, biological process unknown, cellular process, development, obsolete, physiological processes, viral life cycle, etc.) within that category. Optional indicators may include, but are not limited to, the number of subcategories under each category. The headings are followed by selectable species designations (e.g., human, mouse, etc.), which the user can activate, resulting in a search results window as described above.

The search results windows also contains hyperlinks (124 (a) and (b)) which may lead to another WWW site (126), or another place within the same browser (121). In the exemplified system, after a clone has been selected, the user can click the hyperlink in the Clone ID field (124 (a)) which leads to an electronic (ORF) card for the selected clone (123). The card may contain headings such as gene information, open reading frame (ORF) information, clone information, protein information, single nucleotide polymorphism information, and genomic links. In a preferred system, the headings are followed by fields containing hyperlinks to both commercial and private databases (e.g., gov't, universities, consortiums, etc. (126)) which provide further information regarding the category as denoted by the heading.

The Ontology database is regularly updated by manual inputting of new data or by tracking using a Web robot to search the World Wide Web for such new data (e.g., see U.S. Pat. No. 6,718,363).

In one aspect, a preference database may be generated to contain profile data on a user. In a related aspect, a type of device for building a preference database is a passive one from the standpoint of the user. The user merely makes choices (e.g., menu choice in a browser built into a reader) in the normal fashion and the system gradually builds a personal preference database by extracting a model of the user's behavior from the choices. It then uses the model to make predictions about what products or services the user would prefer in the future or draws inferences to classify the user (e.g., an industrial scientist or an academic scientist). This extraction process can follow simple algorithms, such as identifying apparent preferences by detecting repeated requests for the same product or service, or it can be a sophisticated machine-learning process such as a decision-tree technique with a large number of inputs (degrees of freedom). Such models, generally speaking, look for patterns in the user's interaction behavior (i.e., interaction with a UI [user interface] for making selections). Such a database can also be used to control inventory, marketing, manufacturing, send warnings or notices to sales staff, shipping and/or security, IT maintenance, promotions, etc. Further, the database can be a trigger to send such notification by, for example, e-mail or other forms of communication (i.e., electronic or non-electronic means).

As stated above, the Search Results window also contains a GUI (e.g., check box, 606) that can be activated to purchase selected items identified in the search (FIG. 11). The button 606, once activated, loads a shopping cart page which displays the item, quantity ordered, price and total for the amount of product ordered. Further, the page contains offers, services and advertisements that might be helpful to the user. The user may then cancel order (clear cart), recalculate order based on any discounts available, or proceed to checkout by activating the appropriate GUI (e.g., button).

Once the appropriate GUI is activated, a new web page is loaded and the user is directed to input user specific information for purchase and tracking in a customer field (dialog box).

While various embodiments of the present invention have been described above, it should be understood that they have been presented by way of example only, and not limitation. For example, a variety of programming languages can be used to implement the present invention, such a well-known JAVA programming language, C++ programming language, C programming language, C# or any combination thereof. Thus, the breadth and scope of the present invention should not be limited by any of the above-described exemplary embodiments, but should be defined only in accordance with the following claims and their equivalents.

It should also be noted that it does not matter where the databases or other data is stored physically. Networks and Internet may connect one data object to a process just as a data bus connects physical memory or non-volatile storage to a processor. Thus, in this discussion and elsewhere, where no particular mention is made of where data is stored, it is assumed not to matter and that a person of ordinary skill could easily make a suitable decision about where to store data—on a vendor's server, on a reader, at a home network server, on a third party server, etc. Thus, profile data may “follow” a user wherever the user goes. So if a user uses an inputting device (wireless or remote peripheral device) in a public place, the user's personal profile is accessible to the processes the user employs. This assumes appropriate security devices are in place to protect the user's profile data. Also note that it has been assumed in the discussions above, in most cases, that some sort of UI, such as those built into a handheld organizer with a touch screen, is associated with the inputting device discussed to allow data to be displayed and entered. The UI could be part of the device to which the inputting device is attached or with which it is associated or it could be part of the device. The details of the UI are not important, except as otherwise noted, and could be of any suitable type at the discretion of a designer.

The disclosures of all of the recited patents, applications and articles are incorporated herein by reference.

Although the invention has been described with reference to the above examples, it will be understood that modifications and variations are encompassed within the spirit and scope of the invention. Accordingly, the invention is limited only by the following claims.

1. A method of procuring biological content and their products and/or services listed on an electronic inventory file, wherein said inventory file is stored on at least one electronic storage medium which comprises a plurality of files comprising at least one segregated sundry grouping of target items, comprising:
interfacing by at least one user via user terminals and bi-directional communication connections with at least one target item server which accesses said electronic storage medium, wherein extracts comprising at least one associated biological attribute are generated in said server for said target items in said electronic storage medium via an appropriate request;
inputting a request to generate said extracts;
retrieving said extracts;
and
generating a page comprising at least one hierarchical menu output based on such extracts that provides said at least one user at least one subset of said target items stored on said electronic medium,
wherein said at least one menu sorts said target items in said subset into a user accessible file of target items based on an empirical measure of similarity of said associated biological attributes for said sorted target items, and wherein the at least one hierarchical menu output display page identifies said target items sorted into each said file which have at least one associated biological attribute in common to enable said at least one user to differentiate products and/or services of interest stored on said electronic storage medium and to procure said differentiated products by activating an appropriate graphic user interface (GUI) comprising the displayed output page.

2. The method of claim 1, wherein interfacing comprises interaction with one or more browsers.

3. The method of claim 1, wherein the products and/or services are biologically related products and/or services.

4. The method of claim 1, wherein the biologically related products are selected from the group consisting of cloned nucleic acid inserts comprising a structural gene or transcriptional unit, bioassays, labeling and detection dyes, vectors, antibodies, peptides, nucleic acids, enzymes, nucleotides, buffers, cells media, selection molecules, expression systems, lipids, transfection reagents, electrophoresis products, separation columns, affinity compounds, membranes, ORFs, DNA and RNA primers and proteins.

5. The method of claim 1, wherein each searchable extract for the target items further comprises a unique dataset of named annotated text strings having set elements consisting essentially of at least one unique name, at least one base text, at least one biologically related annotation that applies to the base text, and at least one gene ontology category.

6. The method of claim 5, wherein the searchable extract further comprises separate categories containing one or more loci selected from the group consisting of an organism, nucleotide accession number, related accession number, gene name, gene definition, gene symbol, text summary of the gene product, expression profile, mRNA record, references, length of insert in base pairs, nucleic acid sequence, collection name, collection type, vector name, vector antibiotic, host name, Stealth RNA, siRNA, protein accession number, protein record, amino acid sequence, molecular weight, isoelectric point, protease digestion pattern, domain search, predicted secondary structure, known or predicted tertiary and/or quaternary structure, protein model search, Online Mendelian Inheritance in Man (OMIM) data, product data, metabolic pathway data, single nucleotide polymorphism (SNP) data, SNP map data, locus link ID, Unigene ID and genomic alignment data.

7. The method of claim 6, wherein the loci are associated with annotations or objects which provide hyperlinks to at least one internal and/or external database server.

8. The method of claim 1, wherein the interfacing is via a primary Web page browser in an HTML format.

9. The method of claim 1, wherein the request comprises inputting a parsable biological attribute in a sub-window accessible module for entering one or more keywords, one or more annotations, one or more sequences, or one or more unique identification numbers.

10. The method of claim 9, wherein biological attributes are selected from the group consisting of nucleic acid or amino acid sequence, molecular weight, isoelectric point, metabolic and signal pathway participation, restriction map, organism, protease fragments, epitopes, hydropathic profile, tissue distribution, expression pattern, kinetic constants, binding constants, antagonists, agonists, inverse agonists, linkage maps, substrates, ligands, inhibitors, disease association, alleles, homology, biological function, phosphorylation pattern, sub-cellular localization, glycosylation pattern, post-translational modification pattern, motif consensus, crystal structures, pharmacokinetic properties, pharmacologic properties, and toxicologic properties.

11. The method of claim 9, wherein the keyword module and annotation module process word-for-word searching, Boolean searching, proximity searching, phrase searching, truncation searching or a combination thereof.

12. The method of claim 9, wherein the sequence module processes string searches via an in-house or external Blast server.

13. The method of claim 2, wherein the request comprises a keyword jump consisting of accessing a one or more browsers in which the user is shown appropriate content to retrieve records stored on the server via said browsers.

14. The method of claim 13, wherein the appropriate content is a gene ontology category database.

15. The method of claim 14, wherein the ontology category database comprises groupings selected from the group consisting of a biological process, cell component, and molecular function.

16. The method of claim 15, wherein the ontology category database is updated by accessing one or more databases on one or more public servers.

17. The method of claim 16, wherein accessing the one or more public servers comprises using a Web robot to search the World Wide Web.

18. The method of claim 15, wherein the accessed public server databases are selected from the FlyBase (Drosophila), the Saccharomyces Genome Database, Mouse Genome Database (MGD), The Arabidopsis Information Resource database; WormBase; the EBI GOA project; Rat Genome Database (RGD); DictyBase; GeneDB S. pombe; GeneDB for protozoa; Genome Knowledge Base; The Institute for Genomic Research (TIGR); Gramene; (i.e., a comparative mapping resource for monocots); Compugen or the Zebrafish Information Network (ZFIN).

19. The method of claim 13, wherein a tabbed sub-window triggers a page load to access the separate keyword jump browser.

20. The method of claim 13, wherein the separate keyword jump browser is indexed by species and displays a hierarchy structure for user-server interfacing.

21. The method of claim 20, wherein the hierarchy structure is a tree navigation structure.

22. The method of claim 9, wherein the generated menu output display provides matches into a result based on the inputted request.

23. The method of claim 22, wherein any one menu item output on the displayed format page consists essentially of a buy option graphic user interface (GUI) and one or more of the following categories selected from the group consisting of a clone identification number, definition of the expressed product, gene symbol, and accession number.

24. The method of claim 23, wherein when the GUI is activated by the user, such activation triggers the content of the page to be transmitted to a purchase database server, further wherein:
i) the purchase server verifies the transmission to be an order for the product associated with the activated GUI, wherein the verified order is assigned a job number by the purchase server;
ii) the purchase server enters the verified order and stores items selected by the user in a shopping cart database of the purchase server; and
iii) the purchase server updates the shopping cart database in real time to synchronize the shopping cart database with the incoming transmissions.

25. The method of claim 24, wherein a user activating the GUI is identified comprising:
a) comparing the customer information in the purchase server with previously-stored customer database information;
b) indicating if a match exists between a customer name field on the transmitted data and the previously-stored customer database information stored on the purchase server.

26. The method of claim 25, further comprising:
c) adding customer information to the purchase server customer database where the comparing step (a) does not produce a match between the customer name field on the transmitted data and the previously-stored customer database information stored on the purchase server.

27. The method of claim 24, further comprising:
a) associating the transmission to the purchase server with a unique session identifier, including embedding the unique session identifier in a universal resource locator (URL);
b) storing the user activity of the user in the purchase server; and
c) associating user activity with the session identifier.

28. The method of claim 23, wherein the clone identification number and accession number function as hyperlinks to separate servers.

29. The method of claim 28, wherein the separate servers are either in-house servers or public servers.

30. The method of claim 29, wherein the public server is maintained by a government institution, a private institution, a college or university, a consortium or a private individual.

31. A server configuration for procuring biological content and their products and/or services listed on an electronic inventory file, wherein said inventory file is stored on at least one electronic storage medium which comprises a plurality of files comprising at least one segregated sundry grouping of target items, comprising:
interfacing by at least one user via user terminals and bi-directional communication connections with at least one target item server which accesses said electronic storage medium, wherein extracts comprising at least
one associated biological attribute are generated in said server for said target items in said electronic storage medium via an appropriate request;
inputting a request to generate said extracts;
retrieving said extracts;
and
generating a page comprising at least one hierarchical menu output based on such extracts that provides said at least one user at least one subset of said target items stored on said electronic medium,
wherein said at least one menu sorts said target items in said subset into a user accessible file of target items based on an empirical measure of similarity of said associated biological attributes for said sorted target items, and wherein the at least one hierarchical menu output display page identifies said target items sorted into each said file which have at least one associated biological attribute in common to enable said at least one user to differentiate products and/or services of interest stored on said electronic storage medium and to procure said differentiated products by activating an appropriate graphic user interface (GUI) comprising the displayed output page.

32. The method of claim 31, wherein the products and services are biologically related products and services.

33. A method of offering a product or service to a user in a remote location comprising:
i) remotely providing an electronic data server to said user;
ii) receiving an input from said user;
iii) processing said input to produce a first output;
iv) interfacing at least one public consortium database with at least one database proprietary to an offerer of said product or service;
v) selecting a first product or service or a link or description of a first product or service to create an extract; and
vi) outputting said extract to said user.

34. The method according to claim 33, wherein said first service is delivering information to said user.

35. The method according to claim 33, wherein the at least one product is a data product.

36. The method according to claim 33, wherein said user is provided remote access comprising an internet link.

37. The method according to claim 33, wherein said user is provided remote access via electromagnetic wave signal.

38. The method according to claim 33, wherein said user is provided remote access via a metallic conductor.

39. The method according to claim 37, wherein said user is provided remote access via a fiber optic cable.

40. The method according to claim 33, further comprising delivering said product or service to said user.

41. The method according to claim 33, further comprising delivering said product or service to a remote location specified by said user.

42. The method according to claim 33, further comprising packing said at least one product.

43. The method according to claim 33, further comprising generating a message and transmitting said message to a recipient other than said user.

44. The method according to claim 43, wherein said message relates to inventory control.

45. The method according to claim 43, wherein said message relates to a manufacturing request or schedule.

46. The method according to claim 43, wherein said message relates to compliance with an internal corporate procedure or regulation.

47. The method according to claim 43, wherein said message relates to governmental procedure or regulation.

48. The method according to claim 43, wherein said message relates to financial control.

49. The method according to claim 43, wherein said message is transmitted to a sales representative.

50. The method according to claim 43, wherein said message is incorporated into a database tracking user activity relating to an offering.

51. The method according to claim 33, further comprising receiving a second input from said user.

52. The method according to claim 51, wherein said second input is in response to said first output.

53. The method according to claim 52, further comprising selecting a second product or service.


SEQUENCE LISTING (4 sequences)

SEQ ID NO: 1 - DNA, 786 bp, Homo sapiens
atggcagaatcccacctgcagtcatccctcatcacagcctcacagtttttcgagatctggctccatttcgacgctgacggaagtggttacctggaaggaaaggagctgcagaacttgatccaggagctccagcaggcgcgaaagaaggctggattggagttatcacctgaaatgaaaacttttgtggatcagtatgggcaaagagatgatggaaaaataggaattgtagagttggctcacgtattacccacagaagagaatttcctgctgctcttccgatgccagcagctgaagtcctgtgaggaattcatgaagacatggagaaaatatgatactgaccacagtggcttcatagaaactgaggagcttaagaactttctaaaggacctgctagaaaaagcaaacaagactgttgatgacacaaaattagccgagtatacagacctaatgctgaaactatttgattcaaataatgatgggaagctggaattaactgagatggccaggttactaccagtgcaggagaattttcttcttaaattccagggaatcaaaatgtgtgggaaagagttcaataaggcttttgagctgtatgatcaggacggcaatggatacatagatgaaaatgaactggatgctttactgaaggatctgtgcgagaagaataaacaggatctggatattaataatattacaacatacaagaagaacataatggctttgtcggatggagggaagctgtaccgaacggatcttgctcttattctctgtgctggggataactag

SEQ ID NO: 2 - DNA, 786 bp, Homo sapiens; identical to SEQ ID NO: 1

SEQ ID NO: 3 - DNA, 786 bp, Mus musculus; identical to SEQ ID NO: 1

SEQ ID NO: 4 - DNA, 786 bp, Mus musculus
atggcagaatcccacctgcagtcatctctgatcacagcctcacagttttttgagatctggcttcatttcgacgctgacggaagtggttacctggaaggaaaggagctgcagaacttgatccaggagcttctgcaggcgcgaaagaaggctggattggagctatcaccggaaatgaaatcctttgtggatcaatatggacagagagatgatggaaaaataggaattgtagagttggctcacgtcttacccacagaagagaatttcttgctgctctttcgatgccagcaactgaagtcctgcgaggaattcatgaagacttggagaaagtatgatactgaccacagcggcttcatcgaaaccgaggaacttaagaactttctaaaggacctactagagaaagcaaacaagactgtggatgatacaaaactagcagagtacacagacctcatgctgaaactatttgattcaaataatgacggaaagctggaactgacagagatggccaggttactaccagtgcaggaaaatttccttcttaaattccagggaatcaaaatgtgtgggaaagagttcaataaggcttttgagttatatgatcaggatggcaacggatacatagatgaaaatgagctggatgctttgctgaaagatctgtgtgagaagaacaaacaggaattggatattaacaatattactacatacaagaagaacataatggccttgtcggatggagggaagctgtaccgaacagaccttgctcttattctttctgctggagacaactag